(19) United States
(12) Patent Application Publication
(10) Pub. No.: US 2008/0108804 A1
(43) Pub. Date: May 8, 2008

(54) Method for modifying RNAS and preparing DNAS from RNAS

(75) Inventors: Yoshihide Hayashizaki, Ibaraki (JP); Piero Carninci, Saitama (JP); Matthias Harbers, Kanagawa (JP)

Correspondence Address: FOLEY AND LARDNER LLP;SUITE 500, 3000 K STREET NW, WASHINGTON, DC, 20007, US

(73) Assignee: KABUSHIKI KAISHA DNAFORM

(21) Appl. No.: 11/591,682

(22) Filed: Nov. 2, 2006

(51) Int. Cl. C07H 21/04

(52) U.S. Cl. 536/25.3

(57) ABSTRACT

A method is disclosed for the modification of an end of RNA molecules and the use of such modified RNA molecules in cDNA synthesis for the purpose of cloning, detection, sequencing, and amplification of parts of the RNAs, the entire RNAs, or any cDNAs derived from such modified RNAs. The invention relates further to the amplification and the identification of nucleic acid molecules for the purpose of single molecule detection and/or high-throughput sequencing. In addition, a method is provided for the preparation of pooled samples that contains molecules each of which is marked by an “Identifier Sequence” for its origin. The invention facilitates studies on biological systems and analysis of genes expressed therein.

### BACKGROUND ART

Genomes contain the essential genetic information for development and homeostasis of any living organisms. For an understanding of biological phenomena, knowledge is required on how genetic information is utilized in a cell or tissue at a given time point. Many cases are known where mistakes in the utilization of genetic information and related regulatory pass ways or within the expressed genetic information cause diseases in human, plant and animal. The RNA expression can be very different in individual cells in a given tissue or in an entire organism. It is therefore desirable to develop a novel method that enables the preparation and capture of RNA and DNA molecules from a limited number of cells, so that even individual cells within a tissue can be analyzed for their RNA expression, promoter usage and expressed genomic information. New directions in the field of life science are addressing such needs. Novel methodologies are being developed for the capture and analysis of individual DNA and/or RNA molecules, and for the understanding of entire biological systems as, for example, in gene network studies.

### Gene Expression Analysis

Different methods are used for expression profiling and annotation of transcripts. Briefly, large-scale expression studies nowadays use approaches based either on in situ hybridization using, e.g., microarrays, or on high-throughput sequencing of short tags, e.g. SAGE, CAGE, MPSS. Such studies may further be combined with classical approaches like RT-PCR or Northern Blotting to address expression levels of individual genes.

High-throughput expression profiling is commonly done by so-called DNA microarrays (Jordan B., DNA Microarrays: Gene Expression Applications, Springer-Verlag, Berlin Heidelberg New York, 2001; Schena A, DNA Microarrays, A Practical Approach, Oxford University Press, Oxford 1999, both of which are hereby incorporated herein by reference). For such experiments, specific probes representing certain individual genes or transcripts are placed on a support and put in hybridizing conditions with a variety of DNA molecules. Positive signals are obtained if a probe on the support reacts with a molecule present in the sample. Such experiments allow the parallel analysis of a large number of genes or transcripts. However, this approach is limited by the fact that only genes or transcripts can be studied, and they had to be initially identified by other experimental means. Such means include cDNA libraries, partial sequence tags and/or results obtained from computer predictions. In the future, the concept of tiling arrays may also allow for an unbiased expression profiling in organisms for which genomic sequence information is available (Kapranov P. et al., Science 296, 916-919 (2002), hereby incorporated herein by reference), although for tiling arrays interpretation is difficult with respect to the nature of the transcripts detected in the experiment.

Due to the limitations of DNA microarray experiments, alternative approaches are in use for gene discovery and expression profiling based on partial sequences or tags obtained from a plurality of RNA samples. The so-called SAGE (Serial Analysis of Gene Expression) method is known as an efficient method for obtaining partial information on the base sequence of an RNA molecule (Velculescu V. E. et at., Science 270, 484-487 (1995), hereby incorporated herein by reference). To achieve high throughput in tag sequencing, DNA concatemers are formed by ligating multiple short DNA fragments (initially about 10 bp in length) containing information on the base sequences at the 3′-end of multiple RNA molecules. A one-pass sequencing read of such a concatemer can determine the base sequences of many tags, i.e., different RNA molecules, within a DNA concatemer. Recently an improved version of SAGE, the so-called LongSAGE, has been published so as to allow for the cloning of longer SAGE tags (Saha S. et al., Nat. Biotechnol. 20, 508-12 (2002); and US patent applications 20030008290 and 20030049653, all of which are hereby incorporated herein by reference). The concept has been further expanded by the so-called “SuperSAGE” method providing sequencing tags of some 25 bp in length (Matsumura, H. et al., Cell. Microbiol. 7, 11-18 (2005), hereby incorporated herein by reference). The SAGE method is currently in wide use as an important method for analyzing genes expressed in specific cells, tissues or organisms, and SAGE tags are available for reference in the public domain, e.g., at http://cgap.nci.nih.gov/SAGE. More information about recent developments in the SAGE field can be found in Wang, S. M.: SAGE: Current Technologies and Applications, Horizon Bioscience, Norwich, 2005, hereby incorporated herein by reference.

U.S. Pat. Nos. 6,352,828, 6,306,597, 6,280,935, 6,265,163, and 5,695,934, all of which are hereby incorporated herein by reference, disclose different approaches for the high-throughput sequencing of short sequence tags, also denoted as Massively Parallel Signature Sequencing or “MPSS”. As described in more detail in Brenner S., et al., Nat. Biotechnol. 18, 630-634 (2000), and Brenner S., et al., Proc. Natl. Acad. Sci. USA 97, 1655-1670 (2000), both of which are hereby incorporated herein by reference, short sequences from the 3′-end of transcripts are obtained in a highly parallel manner by performing cycles with different enzymatic reactions on a single layer of beads. In WO03/091416, hereby incorporated herein by reference, modifications to the aforementioned approach are disclosed to also make the sequencing of short sequences possible from the 5′-end of transcripts. However, in its initial form MPSS was quite limited by the short read length of signature tags.

The shift from 3′-end related information to 5′-end related information, although technically more demanding, is a mandatory move to link expressed information to a regulatory principle which causes the transcriptional event. Common regulatory elements in the control of gene expression are located in the proximity of the 5′-end of a transcript in the so-called promoter regions of a given gene. Due to alternative promoter usage and rearrangements within primary transcripts due to RNA processing and splicing, for most transcripts in higher organisms, promoter regions cannot be identified from information derived from the 3′-end. Hence new approaches have been developed to obtain specifically sequence tags from 5′-ends of transcripts. Such an approach has been disclosed in PCT/JP03/07514, Shiraki T. et al., Prog. Natl. Acad. Sci. USA 100, 15776-15781 (2003), Kodzius R. et al. Nature Methods 3, 211-222 (2006), and US Patent Application 20050250100, all of which are hereby incorporated herein by reference. This so-called CAGE (Cap-Analysis-Gene-Expression) approach allows for the cloning of 5′-end-specific tags into concatemers in a way similar to the SAGE technology. The so-called CAGE tags not only enable the detection of transcripts and their expression profiling, but further provide information on transcriptional start sites to allow for mechanistic studies on the regulation of transcription or the higher annotation of transcripts. Similar approaches for the cloning of concatemers comprising 5′-end specific sequence information have lately also been published by a number of other laboratories, such as in Hwang B. J. et al., Proc. Natl. Acad. Sci. USA 101, 1650-1655 (2004); Hashimoto S. et al., Nat. Biotechnol. 22, 1146-1149 (2004); Zhang Z. and Dietrich F. S., Nuc. Acids Res. 33, 2838-2851 (2005); and Wei C. L. et al. Proc. Natl. Acad. Sci. USA 101, 11701-11706 (2004), all of which are hereby incorporated herein by reference. All those approaches are distinct by the technical means on how the capturing of true 5′-ends is achieved, e.g., by applying the so-called Cap-Trapper or Oligo-Capping methods further outlined below. Further information on the value of 5′-end related tags can be found in Harbers M. and Carninci P., Nature Methods. 2, 495-502 (2005), hereby incorporated herein by reference.

The aforementioned approaches are still limited with respect to the throughput of tag sequencing. In addition, they require many manipulation steps that can cause mistakes in the sequence information obtained from the concatemers. In particular, amplification steps can cause artifacts as well as a bias in the tag frequencies due to distinct amplification rates for individual DNA fragments. To solve these limitations, future directions have to target at direct capturing of DNA and/or RNA molecules for direct analysis so as to omit unnecessarily complicated manipulations and cloning steps, and at a much higher throughput in data acquisition. Recent developments in the field will open up such new avenues to obtain sequence information at a much higher throughput than presently possible by the classical approaches.

### Sequencing Technologies

The ability to read and decode the genetic code has been one of the greatest breakthroughs in life sciences. The sequencing technologies have become the key to obtaining genetic information. A person skilled in the art knows different approaches for obtaining sequence information including, but not limited to, those described by Sambrook J. and Russuell D. W., Molecular Cloning, A Laboratory Manual, Cold Spring Harbor Laboratory Press, New York, 2001, hereby incorporated herein by reference, like the classic Sanger and Maxim-Gilbert sequencing methods. In recent years, new approaches for high throughput sequencing have been developed. They make use of electrophoresis, sequencing by hybridization, sequencing by synthesis, MPSS sequencing, and non-enzymatic single molecule sequencing. For example, sequencing by hybridization makes use of high-density microarray platforms which have the hybridization patterns to a set of oligonucleotides of defined sequences presented thereon and allow for de novo sequencing of unknown sequences. Perlegen, for example, has used such approaches for the analysis of point mutations within the human genome. Alternatively, sequencing by synthesis can be performed by having a polymerase incorporate nucleotides during the extension of a DNA molecule along a DNA template. When performed on a surface, the incorporation of an individual nucleotide at a defined location can be monitored, and the subsequent order of incorporating nucleotides at a defined location determines the sequence of the nucleic acid molecule at the location. Since a very large number of extension reactions can be performed within one reactor, e.g., on the surface of a glass slide or on a bead array, the approach enables highly parallel sequencing of over 100,000 to 1,000,000 samples per reaction depending on the equipment used. Of particular interest for applying the present invention are novel approaches for the detection and sequencing of single DNA molecules as recently reviewed in Metzker M. L., Genome Res. 15, 1767-1776 (2005); Kling J., Nature Biotechnology 23, 1333-1335 (2005); and Shendure J. et al., Nature Review Genetics 5, 335-344 (2004), all of which are hereby incorporated herein by reference. Some of those approaches are subject to commercial applications as offered by companies like 454 Life Sciences at http://www.454.com/, Helicos BioScience at http://www.helicosbio.com/, Solexa at http://www.solexa.com/Company/overview.htm, Visigen at http://www.visigenbio.com/index.html, or GeneoVoxx GmbH at http://www.genovoxx.de/ (the information found at any of the web pages is hereby incorporate herein by reference). These new approaches should make presently umatched sequencing rates possible. Depending on the methodology used, some devices should sequence very short tags of about 20 to 25 bp at an ultra high throughput of over 1,000,000 or more reads per run (e.g. Helicos, and Solexa), and the device from 454 Life Science can realize much longer sequencing reads of over 100 bp at a rate of some 200,000 to 300,000 reads per run.

Isolation of Full-Length cDNA Molecules

The analysis of RNAs from a biological sample greatly benefits from the isolation of full-length RNA molecules and the preparation of cDNAs derived from such full-length RNAs. In particular as outlined above, sequence information from 5′-ends of RNAs enables a wider interpretation of data. Moreover, full-length cDNA technologies are mandatory for the analysis of biological processes related to RNA processing and splicing. Recent data obtained by different experimental approaches show that the complexity of higher organisms is achieved by an alternative use of genomic information. Additional mechanisms for the combinatorial rearrangement and processing of the transcripted information are important for diversification and expansion of the genetic pool (Zavolan M., et al., Genome Res. 13 (2003) 1290-1300, hereby incorporated herein by reference).

Different approaches for the preparation of full-length cDNAs have been described in the literature as summarized by Das, M., et al., Physiol Genomics 6, 57-80 (2001), hereby incorporated herein by reference. Out of those, the Cap-Trapper and Oligo-Capping methods are most frequently used besides other approaches known to a person skilled in the art in the field. These approaches have been instrumental in understanding genome and transcript structures and decoding protein sequences. Moreover, only full-length cDNAs give access to proteins encoded therein, which can be expressed from functional vectors when needed for experimental studies or industrial applications.

To apply the Cap-Trapper method (Carninci P. and Hayashizaki Y., Methods Enzymol. 303, 19-44 (1999); and U.S. Pat. Nos. 5,962,272 and 6,022,715, all of which are hereby incorporated herein by reference), the diol group of the Cap structure is chemically biotinylated to capture mRNA/cDNA hybrids on Streptavidin-coated beads. Remaining single-stranded RNAs including rRNAs and tRNAs, or RNA portions within partly double-stranded DNA-RNA hybrids are destroyed by RNase I digestion, whereas RNA moieties within mRNA/cDNA hybrids are protected against degradation. Full-length enriched cDNAs are then released from the beads by RNA hydrolysis.

The alternative Oligo-Capping method starts from the modification of 5′-ends of mRNAs (Maruyama K. and Sugano S., Gene 138, 171-174 (1994); and Suzuki Y. and Sugano S., Methods Mol. Biol. 221, 73-91 (2003), both of which are hereby incorporated herein by reference). In the first step, un-capped and truncated mRNAs are dephosphorylated at their 5′-ends by a phosphatase, followed by decapping capped mRNAs by treatment with tobacco acid pyrophosphates (TAP). This treatment leaves only full-length mRNAs phosphorylated at their 5′-ends. Therefore, RNA ligase can only attach an oligonucleotide to the 5′-ends of phosporylated full-length mRNAs. The oligonucleotide attached at the 5′-end of mRNA can be used in later manipulations of the cDNAs derived from such modified mRNAs, e.g., for 2^{nd} strand cDNA synthesis.

Alternative approaches to full-length cDNA selection include the use of a Cap-binding protein (Edery, I., et al., Mol. Cell. Biol. 15, 3363-3371 (1995), hereby incorporated herein by reference) and an antibody against the Cap structure (Theissen, H., et al., Embo J 5, 3209-3217 (1986), hereby incorporated herein by reference), the attachment of an oligonucleotide to the Cap-structure (U.S. Pat. Nos. 5,962,272 and 6,022,715, both of which are hereby incorporated herein by reference), or the SMART™ method from Clontech (http://www.clontech.com/clontech/smart/index.shtml, the information provided therein is hereby incorporated herein by reference). However, the SMART™ approach as a Cap-Switching method (Zhu Y. et al., Biotechniques 30, 892-897 (2001), hereby incorporated herein by reference) adds the trinucleotide GGG to the 5′-end of mRNA, which makes it unfavorable for 5′-end tag cloning due to the reduced length of the informative part of the tag, particularly when sequencing approaches can only make very short sequencing reads.

### SUMMARY OF THE INVENTION

The present invention relates to the modification of an RNA molecule or a plurality of RNA molecules to introduce sequence information at its/their 5′-end. The invention relates to the modification of RNA molecules, so that information added to the RNA molecules is used for their manipulation and/or analysis.

The present invention provides an innovative solution on how to obtain RNA and/or DNA molecules or fragments thereof for single molecule detection and sequencing. The present invention offers a new solution on how to capture individual molecules for detection and analysis as needed for new approaches to single molecule detection. The present invention modifies an RNA molecule and a DNA molecule derived therefrom in such a way that sequence information from a specified region of such modified RNA or DNA molecule can be obtained. Therefore, the invention provides a new high-throughput sequencing approach and its use in, for example, expression profiling, transcript characterization, genome annotation, cloning for further analysis, and other classical means. In particular, the invention provides a further method of high value to studies including, but not limited to, expression profiling based on 5′-end specific sequences, which is an essential component of commercial applications, reagents and services including, but not limited to, life science, drug development, diagnostics, or forensic studies.

In one embodiment, the present invention relates to the transcriptional conversion of a native or artificial RNA molecule or a plurality of RNA molecules into cDNAs. Hence, the invention relates to the synthesis and preparation of single-stranded DNA molecules. As such, the invention relates to a method for the isolation of fragments from nucleic acid molecules for the purpose of detection and analysis. Moreover, the invention relates to the conversion of an RNA sample containing one or more nucleic acid molecules of a single kind or plural kinds into DNA molecules.

In another embodiment, the invention relates to the manipulation of nucleic acid molecules so as to prepare nucleic acid molecules in the form of linear single-stranded DNAs. The invention relates to the preparation and manipulation of linear single-stranded DNAs which are transcripts derived from RNAs.

In a different embodiment, the invention provides a method for introducing functional groups at an end of an RNA or DNA molecule. Thus, the invention provides a method for capturing RNA and DNA molecules for analysis and manipulation by means of a functional group. In this embodiment, the invention relates to the isolation of a single nucleic acid molecule for the purpose of analysis and detection or sequencing.

Hence the invention provides a method for preparing a template for single molecule detection and high-throughput sequencing.

In another embodiment, the invention relates to the use of single-stranded DNA molecules for directly obtaining sequence information thereof. Hence the invention relates to obtaining sequence information from defined regions of single-stranded DNA fragments. In one particular embodiment, the 5′-end specific sequence information obtained from a DNA fragment prepared according to the invention relates to the 5′-end sequence of an RNA molecule. Thus, the invention relates to obtaining sequence information from an RNA molecule.

In another different embodiment, the invention provides a method for modifying the opposite ends of a DNA molecule derived from an RNA, where the modifications at the end corresponding to the 5′-end of the RNA and/or the end corresponding to the 3′-end of the RNA introduce a functional group or a group that otherwise has a function for the further manipulation, detection, and analysis of the DNA molecule. In one specific embodiment, a functional group is introduced at the end corresponding to the 3′-end of the RNA to capture the cDNA by binding it to a surface. In one particular embodiment, the 3′-end specific sequence information obtained from a DNA fragment prepared according to the invention relates to the end sequence of an RNA or DNA molecule. In another embodiment, both ends of the cDNA molecule are modified in such a way that the cDNA molecule can be amplified by means of the LAMP process or by means of the rolling circle amplification (RCA) process.

In another embodiment, the invention provides a method for introducing regions of a defined sequence, the so-called “Identifier Sequence,” at the 5′-end of an RNA. Such an Identifier Sequence identifies the origin of a molecule. Hence, with the introduction of an Identifier Sequence, it becomes possible to analyze a pooled sample which comprises samples of different origins. Such different origins may relate to different cell lines, organisms, or tissues used, or they may relate to different developmental stages or various time points within an experimental study. In one embodiment, the Identifier Sequence is part of the sequence obtained from a molecule prepared according to the invention. In one more embodiment, the Identifier Sequence is used to capture a molecule having such an Identifier Sequence to a specific location on a surface for the purpose of detection and analysis or sequencing. In just one more embodiment, the Identifier Sequence is used to prime the sequencing of a molecule among a plurality of molecules in a pooled sample. Similar to the foregoing, the invention provides a method for introducing Identifier Sequences not only at the 5′-end of an RNA, but also at the region of a cDNA equivalent to the 3′-end of the RNA. The use of an Identifier Sequence is not limited to the 5′-end of the RNA, but may be used at either end of a cDNA derived from the RNA depending on experimental requirements.

The invention relates to the sequencing of certain regions of DNA fragments obtained according to the invention for the purpose for their annotation by computational means including their statistical analysis, annotation by means of alignments to reference information, and/or mapping to genomic sequences. Thus, the invention relates to a method for gene discovery, gene identification, gene expression profiling, and their annotation.

In another further embodiment, the invention relates to the sequencing of DNA fragments obtained according to the invention to allow for their annotation by computational means, the readout of Identifier Sequences, and the statistical analysis of sequences, where such sequences are related to regions within genomes. Hence, the invention relates to the characterization of genetic elements within genomes with reference to transcriptional start sites.

In yet another different embodiment, the invention relates to the preparation of hybridization probes from the ends nucleic acid molecules, where such regions would be analyzed by means of in situ hybridization. In a preferred embodiment, the in situ hybridization experiment makes use of a tiling array.

In one more embodiment, the invention relates to the full-length cloning of nucleic acid molecules in such a way that the sequence information obtained from DNA fragments according to the invention is amplified. It is within the scope of the invention to amplify and clone transcripted regions as well as genomic fragments. Such fragments may contain promoter regions.

Thus, the invention provides a method for the analysis of nucleic acid molecules and short fragments thereof as needed, for example, for the characterization of biological samples. Moreover, the invention provides a method for fast and effective manipulation and/or sequencing of RNA and DNA fragments to make use of such fragments in analytical assays, such as single molecule detection. Hence, the invention is in particular suitable for high-throughput sequencing approaches and the parallel detection of RNA or DNA molecules on a solid support.

In a particular embodiment, the invention relates to the construction of a bidirectional template by means of a modified DNA that can be converted into a circular single-stranded DNA molecule. After amplification of the circular single-stranded DNA molecule by means of the RCA reaction, a bidirectional linear single-stranded DNA molecule is obtained that can be directly attached to a defined location on a solid support. The present invention makes it possible to obtain multiple sequencing reads from the same template at a defined location which links different sequencing reads to the same temple. By the use of a bidirectional linear single-stranded DNA molecule as template sequence information from both strands of the modified DNA molecule or both ends of an RNA molecule can be obtained from the same template.

The invention provides a required method for designing and performing analytical assays that can be used in life science studies and diagnostics. Hence, the invention relates to a method for analyzing a biological system or for diagnostics.

The invention also provides a method for designing and manufacturing a kit and reagents to perform the invention as such or in part as needed to satisfy experimental requirements.

### BRIEF DESCRIPTION OF THE DRAWINGS

FIG. 1 is a conceptual drawing for enzymatic modification of an RNA. As outlined in this figure, RNA preparations may contain RNA species marked by the presence of a Cap structure at their 5′-ends of full-length mRNAs. This Cap structure makes them distinct from other truncated RNA species lacking such a Cap structure and having instead a free phosphate group at their 5′-ends. In a series of enzymatic reactions, first the free phosphate groups are removed from the truncated RNAs by means of a phosphatase. In the second reaction, the Cap structures are removed from the full-length mRNAs by means of a pyrophosphatase, opening up a phosphate group in place of the former Cap structure. Only RNA molecules having a phosphate group at their 5′-ends can be modified by the addition of an oligonucleotide that covalently attaches to the RNA molecules by means of an RNA ligase. The oligonucleotide is shown by the hatched portion.

FIG. 2 is a conceptual drawing showing the modification of an RNA by the addition of an oligonucleotide. Following the course of events outlined in FIG. 1, an RNA ligase can be use to attach different kinds of oligonucleotides to an RNA molecule or a plurality of RNA molecules. According to the invention, the oligonucleotide can be a homopolymer made of desoxyribonucleoties or DNA oligonucleotide, or made of ribonucleoties or RNA oligonucleotide (panel A); a heteropolymer made of desoxyribonucleoties and ribonucleoties or DNA/RNA oligonucleotide (panel B); or can be any kind of homopolymer or heteropolymer having a functional group (indicated by an “F” in panel C).

FIG. 3 is a conceptual drawing showing an alternative method for the ligation of an oligonucleotide to the 5′-end of RNA. As outlined in FIGS. 1 and 2, an RNA Ligase can ligate an oligonucleotide to the 5′-end of a phosphorylated RNA. However, such a reaction may be dependent on a ribonucleotide at the 3′-end of the oligonucleotide, but does not have any sequence specificity, and any oligonucleotide can be combined with any RNA molecule. To give the reaction sequence specificity, a partly double-stranded oligonucleotide can be used which has an overhanging region hybridizing to sequences at the 5′-end of the RNA molecule. Depending on the directions of the reaction, the overhang can have a random sequence or a defined sequence for targeting specific RNA molecules. Moreover, partly double-stranded oligonucleotides can be used to ligate oligonucleotides to the RNA by means of a DNA ligase. Subsequently, the attachment of suitable primers, treatment with a reverse transcriptase, and RNA digestion yield a cDNA as discussed below in further details with variations.

FIG. 4 is a conceptual drawing showing the first-strand cDNA synthesis by means of random priming. Any modified RNA molecules of a single kind or of different kinds, obtained in accordance with any or all steps described in FIGS. 1 to 3, can be used in a reaction to synthesize a cDNA copy of the RNA template by means of a reverse transriptase. This reaction requires primers that can hybridize to the RNA template and initiate the DNA synthesis. For the examples shown in this figure, a set of primers is used having a random sequence at their 3′-end (indicated by NNNN) followed by a defined sequence. Any such primer set can be applied to the primer DNA synthesis from a modified RNA template comprising a sequence and/or a functional group derived from an oligonucleotide attached to its 5′-end (panels A to C).

FIG. 5 is a conceptual drawing showing the first-strand cDNA synthesis by means of an oligo-dT primer. Any modified RNA molecules of a single kind or different kinds obtained in accordance with any or all steps described in FIGS. 1 to 3 can be used to synthesize a cDNA copy of the RNA template by means of a reverse transcriptase. This reaction requires primers that can hybridize to the RNA template and initiate the DNA synthesis. For the examples shown in this figure, an oligo-dT primer is used that can hybridize to the polyA tail commonly found at the 3′-end of many mRNA species. An oligo-dT primer can be applied to prime DNA synthesis from a modified RNA template comprising a sequence and/or a functional group derived from an oligonucleotide attached to its 5′-end (panels A to C).

FIG. 6 is a conceptual drawing showing the RNA removal from DNA/RNA hybrids. The synthesis of a DNA from an RNA template as described in any of FIGS. 4 and 5 leads to the formation of a double-stranded DNA/RNA molecule. The RNA portion within any such double-stranded DNA/RNA molecule can be removed by means of an RNA degrading enzyme or changes in the pH of the reaction buffer. For example the enzyme RNase H specifically digests RNA within double-stranded DNA/RNA molecules, making it a preferable enzyme to practice the invention. Any such treatment on a double-stranded DNA/RNA molecule releases the DNA strand as a single-stranded DNA molecule (panel A) or as a partly double-stranded DNA molecule. The partly double-stranded DNA molecule has parts of the oligonucleotide added to the RNA molecule in the previous steps (panels B to D). Although depicted in the figure for cDNAs obtained by means of an oligo-dT primer, the removal of RNA described here applies to any kind of cDNAs regardless of priming used for the reverse transcription reaction.

FIG. 7 is a conceptual drawing showing the application of DNA molecules obtained by the invention. DNA templates obtained by means of the invention and as depicted in any of FIGS. 1 to 6 may be distinct in their features depending on the nature of the oligonucleotide added to the RNA species at the early stages shown, for example, in FIGS. 2 and 3. The principle structure and their applications are outlined in panels A to D.

FIG. 8 is a conceptual drawing showing the introduction of a functional group at the 3′-end of a cDNA. In accordance with the steps outlined in FIGS. 4 and 5 a common oligo-dT primer or a set of random primers are needed to prime the first-strand cDNA synthesis from an RNA template. Such primers may be comprised of a region having complementary sequence to sequence information within the RNA template, and may comprise other sequence information designed for later use in manipulation of cDNAs. They may further include a functional group attached to such primer as indicated by “F” in the figure. Such functional group can be incorporated into the cDNA regardless of the use of random primers (panel A) or the use of an oligo-dT primer (panel B). Hence, the invention provides a method for preparing cDNA fragments having modified ends.

FIG. 9 is a conceptual drawing showing the capture of modified DNA fragments. In accordance with any of the steps outlined in FIGS. 1 to 7, the invention provides a method for introducing a functional group at a position equal to the 5′-end of an RNA molecule. In addition, in accordance with any of the steps outlined in FIG. 8, the invention provides a method for introducing a functional group at a position equal to the 3′-end of an RNA molecule. The invention also provides a method for introducing a functional group at either end of a cDNA derived from an RNA. When the functional group, indicated by “F” in the drawing, as attached to the cDNA, has a binding affinity to another molecule as indicated by an open clamp in the drawing, the functional group can be used to capture the modified cDNA and attach the modified cDNA to a surface. Panel A depicts the principles of having a partly double-stranded cDNA molecule attached to a surface by means of an interaction of the functional group with a binding partner on the surface. The partly double-stranded region of the cDNA molecule enables the sequencing of the cDNA fragments from the end equal to the 5′-end of RNA. Panel B depicts the principles of having a single-stranded cDNA molecule attached to a surface by means of an interaction of the functional group with a binding partner on the surface. While the cDNA is attached to the surface at a position equal to the 3′-end of RNA, external primers may be used to determine the position from which the cDNA molecule is sequenced. Panel C depicts the principles of having a single-stranded cDNA molecule attached to a surface by means of hybridization to an oligonucleotide or a primer bound to the surface. The primer on the surface can determine which cDNA fragments can be attached to the surface based on complementarity of sequences in the cDNA fragments to the primer on the surface. Moreover, the primer on the surface determines the position from which the cDNA molecule is sequenced.

FIG. 10 is a conceptual drawing showing the introduction of hairpin structures. In accordance with any of the steps outlined in FIGS. 1 to 7, the invention provides a method for introducing an oligonucleotide at a position equal to the 5′-end of an RNA. In addition, in accordance with any of the steps outlined in FIG. 8, the invention provides a method for introducing an oligonucleotide at a position equal to the 3′-end of an RNA. The invention provides a method for introducing specific sequences at either end of a cDNA derived from an RNA so that the cDNA fragment has modified ends. For the processes depicted in this figure, the sequences introduced by means of the invention have the ability to form hairpin structures in which singe-stranded DNA molecules fold into such a configuration that complimentary sequences within the single-stranded DNA molecule form a double-stranded region with a closed loop at one end. Applying the methods disclosed herein, a DNA molecule derived from an RNA can be modified in such a way that it has hairpin structures at opposite ends, regardless whether the cDNA synthesis has been primed by random primers (panel A) or an oligo-dT primer (panel B).

FIG. 11 is a conceptual drawing showing the amplification of modified DNA fragments. A DNA fragment prepared in accordance with the steps outlined in FIG. 10 can be amplified by making use of the hairpin structures at the two opposite ends. As depicted in panel A, a single-stranded DNA fragment having loop structures at the opposite ends is a template for Loop-Mediated Isothermal Amplification, so-called the LAMP method, as disclosed in Notomi T. et al., Nuc. Acids Res. 8, e63 (2000), hereby incorporated herein by reference. A DNA fragment prepared according to the invention can be amplified in such a way that a polymer having repetitive sequences is obtained, and the loop structures within such a polymer can be used to prime the extension of the amplification reaction or can be used to drive a sequence reaction. As depicted in panel B, a DNA fragment having loop structures at each end can be converted into a circular single-stranded DNA molecule by steps comprising a first reaction in which the free 3′-end of one hairpin structure is extended by means of a DNA polymerase lacking any exonuclease and strand-displacement activities. Due to the lack of a strand displacement activity the DNA polymerase will stop when reaching the 5′-end of the opposite hairpin structure. In a second reaction step, the open ends of the single-stranded DNA molecule are ligated to each other to form a circular DNA molecule. Circular single-stranded DNA molecules can be amplified by means of the rolling circle amplification method or the RCA method. A person skilled in the art in the field knows many different applications and modification of the RCA method. For further reference on the RCA method refer to the following review articles: Gusev, Y. et al., American J. Pathology 159, 63-69 (2001), or Zhang D. et al. Clin. Chim. Acta, 363, 61-70 (2006), both of which are hereby incorporated herein by reference. Hence, a DNA fragment prepared according to the invention can be amplified in such a way that a linear polymer of repetitive sequences is obtained. Such a polymer contains at least two copies of the sequence of the initial RNA, so that the two sequences are complementary to each other. Such a polymer also contains sequences that can be used drive a sequence reaction on either of the two sequences derived from the initial RNA.

FIG. 12 is a conceptual drawing showing the introduction of identifier sequences and pooled samples. Any of the steps outlined in FIGS. 1 and 2 can be used to introduce an oligonucleotide at the 5′-end of a full-length mRNA. Such an oligonucleotide may carry regions having sequence information, so-called “Identifier Sequence”, that can be used to identify the origin of the modified RNA and/or any DNA derived from the RNA in a pooled sample. The pooled sample is obtained by mixing different RNA samples having different Identifier Sequences (Panel B). Moreover, the Identifier Sequence may be used for the specific capturing of single molecules for the detection and analysis, or for priming sequencing reactions for selected samples within the Pooled Sample. As such Identifier Sequences can be used to identify the origin of individual RNAs or DNAs derived from the RNAs by determining the Identifier Sequence in a sequence reaction or by selective capturing of molecules having the Identifier Sequence or sequences complementary to the Identifier Sequence at a defined location on a surface (Panel C).

### DETAILED DESCRIPTION OF THE INVENTION

The invention encompasses a method for handling single-stranded as well as double-stranded nucleic acids in the form of linear and circular nucleic acid molecules. Double-stranded DNA means any nucleic acid molecules each of which is composed of two polymers formed by deoxyribonucleotides and in which the two polymers have substantially complementary sequences to each other allowing for their association to form a dimeric molecule. The two polymers are bound to each other by specific hydrogen bonds between matching base pairs within the deoxyribonucleotides. Any DNA molecule composed only of one polymer chain formed by two or more deoxyribonucleotides having no matching complementary DNA molecule to associate with is considered to be a single-stranded DNA molecule for the purpose of the invention, even if such a molecule may form secondary structures comprising double-stranded DNA portions. As used interchangeably herein, the terms “nucleic acid molecule(s)” and “polynucleotide(s)” include RNA or DNA regardless of single or double-stranded, coding or non-coding, complementary or not, and sense or antisense, and also include hybrid sequences thereof. In particular, they encompass genomic DNAs and complementary DNAs, which may be transcribed or untranscribed, spliced or unspliced, incompletely spliced or processed, independent from its origin, cloned from a biological material, or obtained by means of synthesis. RNAs for the purpose of the invention are considered a single-stranded nucleic acid molecule even if such a molecule may form secondary structures comprising double-stranded RNA portions. In particular, RNAs encompass for the purpose of the invention any form of nucleic acid molecules comprising ribonucleotides, and do not relate to a particular sequence or origin. Thus, RNAs may be transcribed in vivo or in vitro by artificial systems or untranscribed, spliced or unspliced, incompletely spliced or processed, independent from its natural origin or derived from artificially designed templates. They may include mRNA, tRNA, rRNA, miRNA, siRNA, RNAi obtained by means of synthesis, or any mixture thereof. RNAs may derive from biological samples or more specifically from fluids of a biological origin, such as blood or serum. For instance, it may contain viral RNA or other potential parasites from the blood of an individual human; or the RNA may be obtained from purified cells, including flow-sorted cells from dissected tissue, where cells may be labeled with a selectable fluorescent antibody for cell sorting, or labeled by the transgenic expression of a marker such as the green fluorescent protein (GFP), using methods known to a person skilled in the art of the field. Alternatively, these cells are selected based on their morphology or by laser capture micro dissection. More precisely, the expressions “DNA”, “RNA”, “nucleic acid”, and “sequence” encompass nucleic acid materials themselves and are thus not restricted to particular sequence information, vector, phagemid or any other specific nucleic acid molecules. The term “nucleic acid” is also used herein to encompass naturally occurring nucleic acids, artificially synthesized or prepared nucleic acids, any modified nucleic acids into which at least one or more modifications have been introduced by naturally occurring events or through approaches known to a person skilled in the art. Similarly, a “tag” or an “Identifier Sequence” according to the invention can be any region of a nucleic acid molecule as prepared by means of the invention. The term “tag” or “Identifier Sequence” as used herein encompasses any nucleic acids fragment, no mater whether it comes from a naturally occurring source, or it is artificially synthesized or prepared. It may also encompass any modified nucleic acids into which at least one modification has been introduced by naturally occurring events or through approaches known to a person skilled in the art. Furthermore, the terms “tag” or “Identifier Sequence” do not relate to any particular sequence information or their composition. The terms “purity”, “enriched”, “purification”, “enrichment”, and “selection” are used interchangeably herein and do not require absolute purity or enrichment of a product. The terms “specific”, “preferable”, or “preferential” are used interchangeably herein and do not require absolute specificity of a DNA or RNA hybridization probe or an enzyme for its substrate, but rather they are intended to signify the possibility that an enzyme may have low or lower affinity compared to other compounds related or unrelated to its substrate. Similarly, the terms used to name an enzyme or an enzymatic activity are to describe the function or activity of such a component and do not require the absolute purity of such a component. Thus, any mixture containing a specific enzyme or enzymes with other components of the same, related or unrelated function are within the scope of the invention. Similarly, DNA or RNA molecules may function in a specific manner as hybridization probes, and as such, they may have “complementary sequences” for the purpose of the invention. DNAs or RNAs having complementary sequences can be used for the detection of a related nucleic acid molecule, even if such a probe and its target molecule may be distinct due to naturally occurring or artificially introduced mutations at different positions. The term “biological samples” includes any kind of material obtained from living organisms including microorganisms, animals, and plants, as well as any kind of infectious particles including viruses and prions, which depend on a host organism for their replication. As such “biological samples” include any kind material obtained from a patient, animal, plant or infectious particle for the purpose of research, development, diagnostics or therapy. Thus, the invention is not limited to the use of any particular nucleic acid molecules or their origin, but the invention provides a general method to be applied to and used for the manipulation and processing of any given nucleic acid. Any such nucleic acid molecules as applied to perform the invention can be obtained or prepared by any method known to a person skilled in the art including, but not limited to, those described in Sambrook J. and Russuell D. W., Molecular Cloning, A Laboratory Manual, Cold Spring Harbor Laboratory Press, New York, 2001, hereby incorporated herein by reference.

The invention relates to methods for the isolation of fragments from nucleic acid molecules for the purpose of analysis and detection. The analysis of a nucleic acid molecule may include, but is not limited to, obtaining part or the entire sequence information of a nucleic acid molecule. A person skilled in the art knows different approaches for obtaining sequence information including, but not limited to, those described in Sambrook J. and Russuell D. W., Molecular Cloning, A Laboratory Manual, Cold Spring Harbor Laboratory Press, New York, 2001, or newly evolving high throughput technologies described in the background art. The invention is not limited to the use for any particular sequencing approach or technology, and it provides a general method for manipulating RNA and DNA for analysis and detection as most suitable for the experimental needs or as appropriate in light of new developments in the field.

For manipulation, detection, or analysis including a sequencing reaction, nucleic acid molecules may be attached or otherwise bound to a solid support. A solid support may be any solid material with which components can be associated directly or indirectly. Such material includes, but is not limited to, acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polyactic acid, polyorthoesters, functionalized silane, polypropylfumerate, collagen, glycosaminoglycans, polyamino acids, or any combination thereof. Solid supports may further include thin films, membranes, bottles, dishes, fibers, woven fibers, shaped polymers such as tubes, particles, beads, microparticles, or any combination thereof.

Thus, the invention relates to the conversion of a sample containing one or more nucleic acid molecules. Such nucleic acid molecules or any mixture of nucleic acid molecules would be converted into DNA. To perform the invention, nucleic acid molecules can be derived from any naturally occurring genomic DNA or RNA sample or from an existing DNA library of artificial origin, or any mixture thereof. The invention is not limited to the use of an individual nucleic acid molecule or any plurality of nucleic acid molecules, but the invention can be performed on an individual nucleic acid molecule or any plurality of nucleic acid molecules regardless whether such molecules would occur in nature, be derived from an exciting library, or be artificially created. Furthermore, the invention can process any nucleic acid molecule regardless of its origin or nature. Thus, it is within the scope of the invention that the nucleic acid molecules could be full-length molecules as compared to naturally occurring nucleic acid molecules, or any fragment thereof. Even furthermore, it can be envisioned that such fragments of nucleic acid molecules may be prepared by a random process or by a targeted dissection of nucleic acid molecules by means of an enzymatic activity with a preference for a certain sequence, or by means which would allow for the fragmentation based on the structure of the nucleic acid molecule including, but not limited to, exons and introns within transcripted regions. Thus, the invention is not restricted to the use of any particular starting material.

The invention relates to the modification of an RNA molecule or a plurality of RNA molecules to introduce sequence information and/or a functional group at the 5′-end of an individual RNA molecule or RNA molecules within a pool of RNA molecules. Such a functional group may comprise 1, 3, 1 to 5, 5 to 10, 10 to 15, 15 to 25, 25 to 35, 35 to 45 or more than 45 nucleotides. Hence, the invention relates to the modification of an RNA in such a way that information added to the RNA molecule is used for the manipulation and/or analysis of the RNA molecule or for the preparation and analysis of the modified RNA.

A person skilled in the art knows about different enzymatic and chemical approaches for the modification of RNA. Preferably, in order to practice the invention, an RNA is modified by enzymatic reactions so that a selective use of different enzymatic activities allows a targeted modification of certain RNA species within groups of RNAs. More preferably, mRNA molecules within total RNA are preferentially targeted for modification to allow for selective enrichment. However, the invention is not limited to the analysis of mRNA but provides a general method for capturing an RNA species for analysis and detection. Here results from recent studies point at entirely new RNA species like miRNA and other short RNA molecules (Alvarez-Garcia I. and Miska E A., Development 135, 4653-4662 (2005), hereby incorporated herein by reference) that could become subject to specific modification and analysis.

To perform the invention, the target RNA is subjected to three conceptually different steps: (1) masking of the non-full-length mRNA molecules, (2) conversion of the Cap structure within molecules into reactive molecules, and (3) attaching the treated RNA molecules to the 5′-end of target RNAs. A standard procedure for adding an RNA oligonucleotide to the 5′-ends of mRNAs is the so-called Oligo-Capping method (Maruyama K. and Sugano S., Gene 138, 171-174 (1994); and Suzuki Y. and Sugano S., Methods Mol. Biol. 221, 73-91 (2003), both of which are hereby incorporated herein by reference), and modifications thereof. RNA preparations from a living organism contain RNA species marked by the presence of a Cap structure at the 5′-ends of full-length mRNAs. This Cap structure makes them distinct from other truncated RNA species lacking such a Cap structure and having instead a free phosphate group at their 5′-ends. The Oligo-Capping approach makes use of the unique feature of full-length mRNAs for selective enrichment. Oligo-Capping comprises a number of enzymatic steps to specifically modify mRNA molecules within a pool of RNAs. In the first enzymatic reaction uncapped RNAs, such as truncated mRNAs, small RNAs, tRNAs, and rRNAs, are dephosphorylated at their 5′-ends by a phosphatase, followed by a second reaction step in which capped mRNAs are decapped by treatment with tobacco acid pyrophosphatase (TAP). This treatment leaves only full-length mRNAs phosphorylated at their 5′-ends. Therefore, in a third enzymatic reaction an RNA ligase can only attach an oligonucleotide to the 5′-ends of phosporylated full-length mRNAs.

For the first reaction step any phosphatase can be use that is able to remove the phosphate group from the 5′-end of RNA. More specifically, the phosphatase can be selected out of a list of the Bacterial Alkaline Phosphatase (BAP), Calf Intestine Alkaline Phosphatase (CIAP), Shrimp Alkaline Phosphatase (SAP), or Antarctic Phosphatase. Similarly different pyrophosphatases may be used to perform the invention, where most commonly the tobacco acid pyrophosphatase (TAP) is used for the removal of the Cap structure. For the RNA ligation step, any RNA Ligase can be used that can ligate an DNA and/or RNA oligonucleotide to phosphorylated RNA. Most commonly the T4 RNA ligase or the Thermo Phage single-stranded DNA ligase is used in this reaction. The Thermo Phage single-stranded DNA Ligase is a commercially available enzyme that can work both on single-stranded DNA and RNA (for more information on the enzyme refer to the product information under http://www.prokaria.com/upload/files/Thermophage-ssDNA-ligase-version-4-2.pdf, hereby incorporated herein by reference). Therefore this enzyme may be preferable to directly ligate an DNA oligonucleotide to RNA. Hence the invention provides a method for directly ligating DNA to RNA so as to prepare a linear heteropolymer composed of desoxyribonucleotides or DNA oligonucleotides and ribonucloetides or RNA or RNA oligonucleotides.

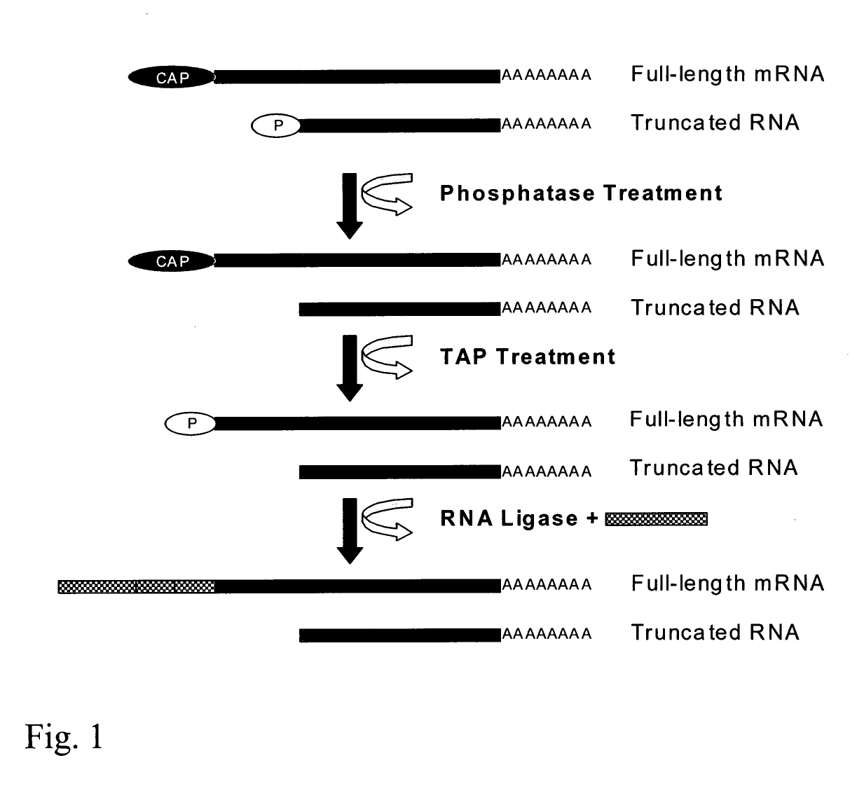

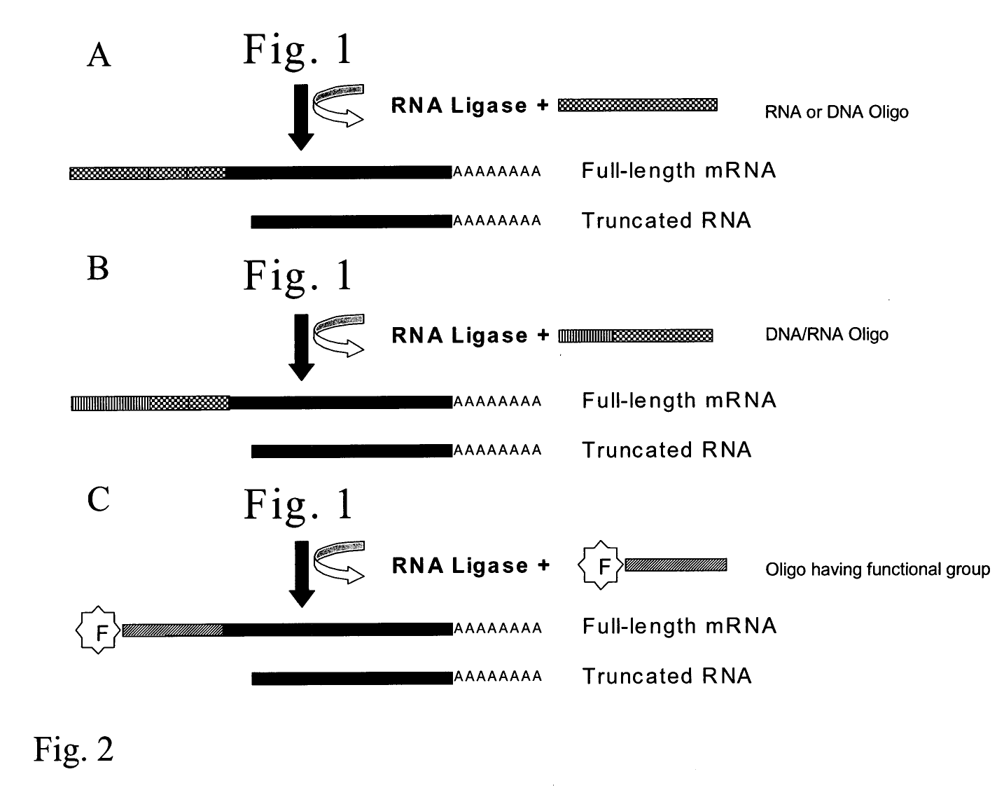

A person skilled in the art knows different modifications of the Oligo-Capping approach that can be used to perform the invention. Most preferably the invention makes use of a procedure where all enzymatic reactions are performed in a single reaction vial as disclosed in patent application JP2006-106770, hereby incorporated herein by reference. In brief, the first reaction step makes use of a phosphatase that can be inactivated by heat treatment, such as Antarctic Phosphatase. After inactivation of the first enzyme in the reaction chain, buffer conditions are changed by the addition of new components suitable for running the TAP reaction. TAP can again be inactivated by heat treatment. Therefore, only another change in the buffer conditions by the addition of additional components and an oligonucleotide is sufficient to perform the ligation of an oligonucleotide to phosphorylated RNA as a final reaction step (compare FIG. 1 for further reference).

In the above, the modification reaction is performed in such a way that mRNA molecules within a pool of RNAs are modified for further manipulation. However, the invention is not restricted to the modification of mRNAs. In a different example, all phosphorylated RNA molecules lacking a Cap structure are directly modified by the ligation of an oligonucleotide to the 5′-end of RNAs. In this example, the invention enables a selective modification of non-mRNA molecules and truncated mRNA molecules. In just a different example of the invention, RNA molecules lacking a Cap structure are modified in a first enzymatic reaction. In one example, only the RNA molecules lacking a Cap structure are modified for manipulation according to the invention. In a different example, the first reaction step is followed by other steps to stepwise modify different RNA molecules. Therefore, in a second enzymatic reaction, the Cap structure of the full-length mRNA molecules is removed by an enzymatic reaction, TAP, to create phosphate groups at the 5′-end of the full-length mRNA molecules. In the last reaction step, an oligonucleotide of the same or different sequences is ligated to the full-length mRNA molecules. In this embodiment, the invention provides a method for adding different oligonucleotides to certain different RNA species within a pool of various RNA molecules.

Following the course of events outlined above and further described in FIG. 1, in the last reaction step an RNA ligase can be use to attached different kinds of oligonucleotides to an RNA molecule or a plurality of RNA molecules. According to the invention, the oligonucleotide can be a homopolymer made of desoxyribonucleoties (DNA oligonucleotide), or of ribonucleoties (RNA oligonucleotide), a heteropolymer made of desoxyribonucleoties and ribonucleoties (DNA/RNA hybrid oligonucleotide), or can be any kind of homopolymer or heteropolymer having a functional group (compare FIG. 2 for reference). The invention is not limited to the use of one specific nuclei acid molecule, but different types of oligonucleotides can be used dependent on the manner in which the modified RNA will be used in different embodiments of the invention. A person skilled in the art will know many DNA and RNA modifications. For example, information on oligonucleotides for the preparation of different modified oligonucleotides is found in the web site of MWG Biotech at http://www.mwg-biotech.com/html/s_synthetic_acids/s_modifications.shtml, the information found therein is hereby incorporated herein by reference. MWG Biotech can provide oligonucleotides having a biotin or a digoxigenin as a functional group at different positions of an oligonucleotide. Moreover, modified oligonucleotides can be obtained having one or more functional groups such as reactive groups for cross linking like the 5′ Aminolink C3/C5/C6/C12, 3′ Aminolink C3/C6/C7, 3′ Aminolink C3/C6/C7, Amino (C2/C6)-dT, Amino C6-dC, Spacer C3/C9 (TEG), Spacer C12/C18 (HEG), or a reduced Thiol modifier. RNA oligonucleotides can be purchased, for example, from Invitrogen under http://www.invitrogen.com/content.cfm?pageid=9900, the information therein is hereby incorporated herein by reference, or Operon under http://www.operon.com/, hereby incorporated herein by reference. Hence the oligonucleotides added to the 5′-end of RNA can be designed for having different functions. In one example, the oligonucleotide has 10 to 25 nucleotides. In a different example, the oligonucleotide has 25 to 50 nucleotides. In just a different example, the oligonucleotide has 50 to 100 nucleotides. In a different example, the oligonucleotide has over 100 nucleotides. The oligonucleotide can be obtained by means of chemical synthesis or can be prepared by an enzymatic reaction. A person skilled in the art knows different DNA-dependent RNA polymerases such as the T3 RNA polymerase, T7 RNA polymerase, or the SP6 RNA polymerase that can be used to prepare RNA molecules from template DNA. An example for a protocol for the preparation of RNA by means of an RNA polymerase can be found on the homepage of Fermentas at http://www.fermentas.com/techinfo/modifyingenzymes/protocols/p_synthstrspecrna.htm, hereby incorporated herein by reference. Moreover, the oligonucleotide can be of natural origin such as ribosomal RNA. Most of the RNA found in preparations of total RNA from any organism is rRNA, and, for example, 18S+28S ribosomal RNA from calf liver can be commercially obtained from Sigma-Aldrich (St. Louis, USA, catalog number R0889). An RNA oligonucleotide, a DNA oligonucleotide or a modified oligonucleotide can be ligated directly to an RNA molecule by means of an RNA ligase as outlined in the above. Hence the reaction conditions are not restricted to the use of a particular ligase, any particular modification of an oligonucleotide, any particular sequence of an oligonucleotide, or any particular oligonucleotide as such.

Most commonly the oligonucleotides are designed to function as “primers” for the introduction of priming sites at 5′-ends of RNAs. Primers may be an oligonucleotide comprising 5, 6, 5 to 10, 10 to 15, 15 to 20, 20 to 30, 30 to 40, 40 to 50 or more than 50 nucleotides. After synthesis of a complementary nucleic acid strand, the 3′-end of the new synthesized second strand will have complementary sequences to the oligonucleotide attached to the 5′-end of RNA. Hence, oligonucleotides having entirely or in part the same sequence as the oligonucleotide added to the 5′-end of RNAs can be used to prime the synthesis of nucleic acid molecules having in part or entirely the same sequence as that of the modified mRNAs. The priming of the second strand can be used, for example, for the preparation of double-stranded or single-stranded DNAs, for DNA or RNA amplification, and for sequencing. Different approaches for the synthesis of a second DNA strand by means of a DNA polymerase can be found in standard textbooks such as Sambrook J. and Russuell D. W., Molecular Cloning, A Laboratory Manual, Cold Spring Harbor Laboratory Press, New York, 2001, hereby incorporated herein by reference. Such DNA polymerases include, but are not limited to, the Klenow fragment of DNA polymerase I, T4 and T7 DNA polymerases, DNA polymerase I, Taq polymerase, Tfl DNA polymerase, Tth DNA polymerase, Tli DNA polymerase, or any other DNA polymerase known in the field. For example, for the preparation of linear single-stranded DNA, various technologies have been developed familiar to a person skilled in the sate of the art in the field. Some approaches use a DNA-polymerase-based synthesis of single-stranded DNA from a DNA or RNA template. In a particular case, the synthesis of a single-stranded DNA can be achieved by the so-called asymmetric PCR reaction, in which the two primers are used at different concentrations. After the rate-limiting primer is exhausted, the reaction switches from the exponential amplification of double-stranded DNA to the linear amplification of the one strand primed by the primer used in excess over the rate-limiting primer. In an alternative approach lambda exonuclease is used to digest the one strand of double-stranded DNA having a 5′-phosphorylated end. Such a template can be prepared in PCR reactions in which only one out of two primers is phosphorylated at the 5′-end. The lambda exonuclease, also denoted as “Strandase™”, is commercially available from Novagen, Madison, USA, and the documentation on its “Strandase™ ssDNA Preparation Kit”, Cat. No. 69202, is hereby incorporated herein by reference. Similarly, the enzyme can also be obtained as lambda exonuclease from Epicentre, Madison, USA (Cat. Nos. LE035H and LE032K). For a number of applications of single-stranded linear DNA, the single-stranded DNA is prepared by means of the PCR reaction in which one of the two primers is specifically tagged. While not limited to it, a biotin label is most frequently applied to separate the strand having a functional group and the second undesired strand from the template DNA. This approach is of value particularly when the strand of interest is supposed to be used as attached to a matrix or any kind of solid support. The immobilized single-stranded DNA can be directly purified on the support and used in detection assays depending on strand specific preparation and isolation of single-stranded DNA or in the preparation of a template for DNA sequencing. One such application includes, but is not limited to, the detection and characterization of SNPs in genomic DNA in, for example, the so-called DASH SNP detection system. This approach is described in US Patent Application No. 2001046670, which is hereby incorporated herein by reference. S. Stahl et al. (Stahl, S. et al, Nucleic Acid Research 16, 3025-3038 (1988), hereby incorporated herein by reference) have found a different application in which biotinylated DNA is used, for example, for sequencing on solid phase. In just another example, in a reaction cycle combining the activities of a reverse transcriptase, RNase H, and a DNA-dependent RNA polymerase a modified RNA molecule can be amplified in accordance with the method published by Guatelli J. C. et al., Proc. Natl. Acad. Sci. USA 87, 1874-1878 (1990), hereby incorporated herein by reference.

In a different embodiment, the oligonucleotides attached to the 5′-end of RNA are designed to have sequence information to enable the manipulation of the RNA molecule or any DNA molecule derived therefrom. A person skilled in the art knows many enzymatic activities that depend on binding to specific sequences or recognition sites. Many such enzymes can be commercially obtained from different suppliers including, but not limited to, FERMENTAS UAB (Vilnius, Lithuania), New England Biolabs Inc. (Beverly, USA), Promega (Madison, USA), Takara (Tokyo, Japan), Roche (Mannheim, Germany), and GE Biosciences (Cardiff, United Kingdom). Commonly restriction endonucleases cut only double-stranded DNA but do not cut single-stranded DNA. Most commonly restriction endonucleases are used to digest DNA molecules at defined locations such as their recognition site or locations in the proximity of their recognition site. In one example the recognition site introduced by an oligonucleotide and attached to the 5′-end of RNA is a restriction site for a class-IIs restriction enzyme. These enzymes cleave outside of their recognition sequence, where, for example, the Class IIs restriction enzyme MmeI cleaves 20/18 base pairs apart from its recognition site. Therefore, MmeI is commonly used for the isolation of short sequencing tags as, for example, in the aforementioned LongSAGE, 5′-SAGE and CAGE approaches. Other applications would make use of restriction endonucleases for the purpose of DNA recombination and cloning known to a person skilled in the art, and further described in Sambrook J. and Russuell D. W., Molecular Cloning, A Laboratory Manual, Cold Spring Harbor Laboratory Press, New York, 2001, hereby incorporated herein by reference. Moreover, a modified or otherwise designed restriction endonuclease can function as a strand-specific nicking enzyme, which cleaves only one DNA strand within its recognition sequence in a double-stranded DNA substrate. Such enzymes include, but are not limited to, the commercially available nucleases N.Bpu 10I (FERMENTAS UAB, Vilnius, Lithuania), N.Bbv C IA, N.Bst NB I and N. Alw I (New England Biolabs Inc, Beverly, USA). Nicking enzymes are of particular interest to create priming sites within double-stranded DNA, which can be used for primer extension reactions toward DNA synthesis and sequencing. An example of a reaction in which one DNA strand within a double-stranded DNA molecule is nicked by an enzymatic activity to create a priming site for DNA synthesis has been described by Walker T. G. et al., Proc. Natl. Acad. Sci. USA 89, 392-396 (1992), hereby incorporated herein by reference. In a different example, the oligonucleotide introduces a recognition site for a RNA polymerase including, but not limited to, the T3 RNA polymerase, T7 RNA polymerase, or SP6 RNA polymerase, all of which are DNA-dependent RNA polymerases with specificity for their respective double-stranded promoters. Starting from the promoters or their recognition sites, they catalyze the 5′-to-3′ synthesis of a complementary RNA from either a single-stranded DNA or double-stranded DNA template. Guatelli J. C. et al. have described an example for the use of a DNA-dependent RNA polymerase in Proc. Natl. Acad. Sci. USA 87, 187401878 (1990), hereby incorporated herein by reference. In a different example, the oligonucleotide may have recognition sites for DNA binding proteins. Many DNA binding proteins are known to a person skilled in the art, which can be of natural occurrence or may have been prepared by means of protein design. Such DNA binding proteins include, but are not limited to, transcription factors, proteins of regulatory function that bind directly or indirectly to recognition sites in genomic DNA. Transcription factors are essential molecules for life and needed for the utilization of genomic information. Every living organism contains a large number of transcription factors. As an example, Kanamori M. et al. have published a database on all the known transcription factors from mouse in Biochem Biophys Res Commun, 322, 787-93 (2004), hereby incorporated herein by reference. Transcription factors are distinct in terms of their affinity to different recognition sites. This specificity can be used for the enrichment of DNA molecules comprising recognition sites for a given transcription factor and/or group of transcription factors. However, the binding specificity of a transcription factor is not limited to binding a certain sequence, as a person skilled in the art will know proteins that rather recognize structures than specific sequences. For example, the transcription factor DAX-1 can bind to different DNA structures as described by Zazopoulos E. et al., Nature 390, 311-315 (1997), hereby incorporated herein by reference. In a different example, a DNA binding protein may bind specifically to single-stranded DNA. Single-stranded-DNA binding proteins including, but not limited to, SSB from E. coli, the product of the phage T4 Gene 32, the adenovirus DBP, an antibody directed against single-stranded DNA, calf thymus UPI, or any mixture thereof. In addition there are proteins that specifically bind to mismatches in double-stranded DNA. This group of proteins includes, but is not limited to, the family of MutS proteins (for reference on the protein family refer to http://www.tigr.org/˜jeisen/MutS/MutS.html, the content of this webpage is hereby incorporated herein by reference), related to a major mismatch repair pathway in E. coli. Where primers are used in primer extension reactions that have a mismatch in their sequence as compared to the complementary sequence or parts thereof attached to the modified RNA or any DNA derived thereof, a MutS proteins or any member of the gene family may be used to specifically enrich double-stranded DNA species having mismatches. In addition MutS or any member of the gene family may be used to block or otherwise manipulate primer extension reactions. Some MutS proteins are commercially available as, for example, Taq MutS from Nippongene (Tokyo, Japan, Code Number 316-04011). In a different example the oligonucleotide may have regions of a given sequence that can be used as an “Identifier Sequence” or “Barcode”. Such a given sequence can be used as an Identifier Sequence to mark the origin of a sample, or it can function as a tag to specifically capture a modified RNA or any DNA derived thereof by means of hybridization to a nucleic acid molecule or the like having complementary sequence to the Identifier Sequence. Such a sequence can also be used as a specific and selective priming site for any of the aforementioned enzymatic reactions. As such, the Identifier Sequence can be a selective priming site for second-strand synthesis by a DNA polymerase, amplification, for example, by means of a PCR reaction, or preparation of a single-stranded DNA. Hence, in combination with the aforementioned method for introducing different oligonucleotides such as Identifier Sequences or recognition sites to different RNA species, the invention provides another method for separately manipulating individual RNA molecules within a plurality of RNA molecules or the total RNA.

In a different embodiment, the invention provides a method for introducing functional groups at the end of an RNA molecule or a variety of RNA molecules. Many different functional groups have an affinity to bind to a binding molecule. A functional group may include, but is not limited to, a reactive group or cross linker suitable to form a covalent bound in a chemical reaction, an amino group, biotin, digoxigenin, antibody, antigen, a protein, a nucleic acid, a nucleic acid binding molecule, or any combination thereof. The functional group and any molecule attached to the functional group can bind to binding molecules which are presented on a matrix. For the purpose of the invention a matrix may be selected from any immobilized form of a reactive group that can be used in a chemical reaction to form a covalent bound, such as avidin, streptavidin, a digoxigenin-binding molecule, an oligonucleotide having a defined sequence, an antibody or its ligand, and a chemical matrix. If the applied functional group is biotin, then the related matrix is avidin or streptavidin. Similarly, when the functional group is digoxigenin, the matrix is a digoxigenin-binding molecule (see Roche Diagnostics GmbH Catalog, the documentation therein is hereby incorporated herein by reference). When the functional group is an oligonucleotide, the matrix is an oligonucleotide having a sequence complementary to that of the functional group, or when the functional group is an antigen, the matrix may be an antibody or an antibody-binding protein such as protein I or protein G. Hence, the invention provides a method for introducing a functional group to an RNA molecule, where such a functional group is attached to the oligonucleotide. Modified oligonucleotides can be commercially obtained from many providers. Most frequently biotin-labeled oligonucleotides are used in the field. For an example of the preparation of different modified oligonucleotides, see the web site of MWG Biotech at http://www.mwg-biotech.com/html/s_synthetic_acids/s_modifications.shtml, the information available therein is hereby incorporated herein by reference. MWG Biotech can provide oligonucleotides having biotin or digoxigenin as a functional group at different positions in an oligonucleotide. Moreover, modified oligonucleotides can be obtained having one or more functional groups such as reactive groups for cross linking like the 5′ Aminolink C3/C5/C6/C12, 3′ Aminolink C3/C6/C7, 3′ Aminolink C3/C6/C7, Amino (C2/C6)-dT, Amino C6-dC, Spacer C3/C9 (TEG), Spacer C12/C18 (HEG), or a reduced Thiol modifier. RNA oligonucleotides can be purchased, for example, from Invitrogen and some information on such available RNA oligonucleotides is available at http://www.invitrogen.com/content.cfm?pageid=9900, This information is hereby incorporated herein by reference. Also, Operon provides some useful information at http://www.operon.com/, and such information is hereby incorporated herein by reference.

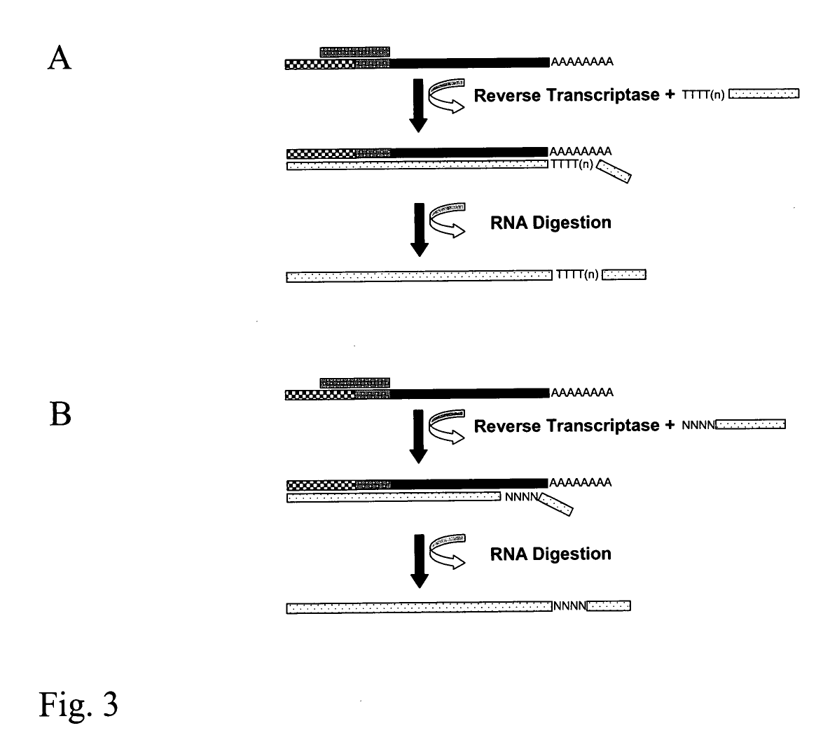

In the aforementioned embodiments, and as further outlined in FIGS. 1 and 2, an RNA ligase is used to ligate an oligonucleotide to the 5′-end of a phosphorylated RNA molecule. Commonly used RNA ligases, like the T4 RNA ligase, are dependent on a ribonucleotide at the 3′-end of the oligonucleotide and may not ligate directly desoxyribonucleotides to RNA. Moreover, the ligation of an oligonucleotide to an RNA molecule is not sequence specific, and any oligonucleotide of a given sequence can be combined with any RNA molecule. An alternative approach has been described by Clepet C. et al. in Nucleic Acids Res. 32, e6 (2004), hereby incorporated herein by reference, where a DNA ligase is used to ligate a double-stranded or partly double-stranded DNA molecule to RNA (so-called RNA-tagging). For example, the T4 DNA ligase can catalyze the ligation of RNA fragments on a DNA template (Kleppe, K. et al., Proc. Natl. Acad. Sci. USA, 67, 68-73 (1970) and Fareed, G. C. et al., J. Biol. Chem., 246, 925-932 (1971), both hereby incorporated herein by reference). The DNA template-mediated ligation reaction can be used to make the ligation reaction sequence specific so as to make it possible to modify an individual RNA molecule. The sequence specificity can be achieved by a partly double-stranded oligonucleotide. Such an oligonucleotide has an overhanging region hybridizing to sequences at the 5′-end of the RNA molecule (compare to FIG. 3). The overhang maybe has a length of 4 to 6 nucleotides or 6 to 8 nucleotides. It may have 8 to 12 nucleotides or more than 12 nucleotides in length. A person skilled in the art knows different approaches using partly double-stranded oligonucleotides in ligation reaction as discussed, for example, by Shibata Y. et al. in Biotechniques, 30, 1250-1254 (2001), hereby incorporated herein by reference. Depending on the directions of the reaction, the overhang can have a random sequence as, for example, used by Shibata et al. in the aforementioned publication or can have a defined sequence for targeting specific RNA molecules. In a different example, the overhang may be created in an enzymatic reaction by using a restriction endonuclease that has a random sequence within its recognition site or cleaves outside of its recognition site. Such enzymes would include, but are not limited to, BstXI (CCANNNNN ↓NTGG), DrdI (GACNNNN ↓NNGTC), BglI (GCCNNN ↓NGGC), BoxI (GACNN ↓NNGTC), BseJI (GATNN ↓NNATC), BseLI (CCNNNN ↓NNGG), CaiI (CAGNNN ↓CTG), CseI (GACGC(5/10)↓), Eam 1105I (GACNNN ↓NNGTC), Eco31I (GGTCTC(1/5)↓), Eco57I (CTGAAG (16/14)↓), Esp3I (CGTCTC(1/5)↓), HpyF10VI (GCNNNN ↓NNGC), LguI (GCTCTTC(1/4)↓), OliI (CACNN ↓NNGTG), PdmI (GAANN ↓NNTTC), PsyI (GACN ↓NNGTC), SfiI (GGCCNNNN ↓NGGCC), SmuI (CCGGC(4/6) ↓), Van91I (CCANNN ↓NTGG), XagI (CCTNN ↓NNNAGG), or their isoschizomers. These enzymes are of particular interest, where random overhangs are prepared from a plurality of nucleic acid molecules. For example, a cDNA library could be constructed in which the cDNA inserts are flanked at their 5′-ends to a linker having a recognition site for one of the aforementioned enzymes. Cleavage of the molecules within such a library would generate a plurality of molecules having random overhangs that are representative for the molecules present within the original cDNA library. Hence, the invention provides a method for targeted modification of individual RNA molecules by means of a DNA template comprising regions having sequences complementary to the oligonucleotide used in the reaction and regions complementary to the 5′-end of an RNA molecule. In one example, those sequences are 5 to 10 nucleotides in length, in a different example those sequences are 10 to 25 nucleotides in length, and in a different example those sequences are longer than 25 nucleotides in length. Regions complementary to the 5′-end of an RNA molecule can be obtained by an experimental means, for example, by the manipulation of a plurality of nucleic acid molecules, or by computational design in combination with chemical synthesis. Information on the sequence of an RNA molecule can be obtained by searches in a public database known to a person skilled in the art such as NCBI at http://www.ncbi.nlm.nih.gov/, EMBL at http://www.ebi.ac.uk/Databases/, or the DDBJ at http://www.ddbj.nig.ac.jp/. In one example, one DNA template is used to target a specific RNA molecule. In a different example, a plurality of DNA templates having in part regions of random sequence are used. In just a different example, a plurality of DNA templates is used having specific sequences. Hence, the invention provides a flexible method for targeted manipulation of RNAs such as mRNA and specific RNA molecules within the total RNA.

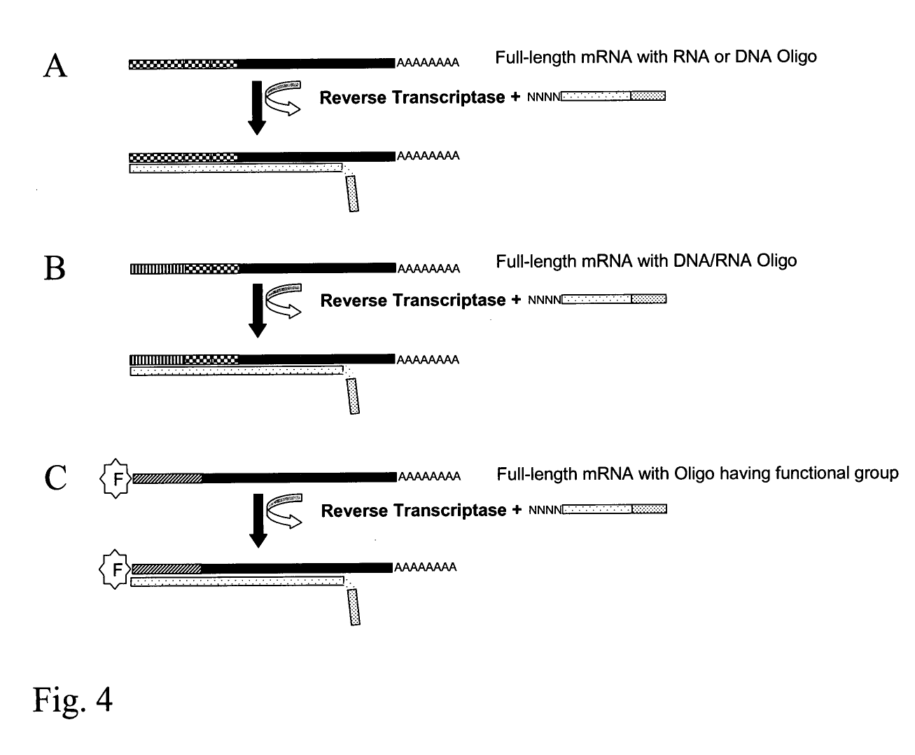

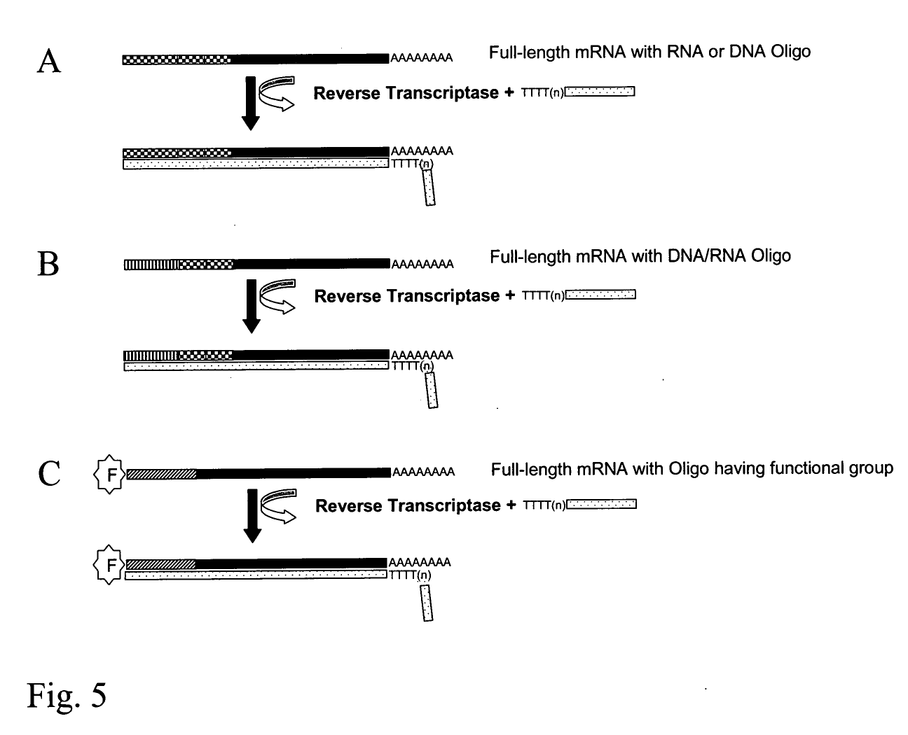

A RNA molecule can be used as a template to prepare a DNA transcript by means of a reverse transcriptase as described in Sambrook J. and Russuell D. W., Molecular Cloning, A Laboratory Manual, Cold Spring Harbor Laboratory Press, New York, 2001, hereby incorporated herein by reference, and a person skilled in the art in the field knows many modifications of the process including different reaction conditions and enzyme modifications. For example, reverse transcriptases include, but are not limited to, AMV reverse transcriptase, M-MLV reverse transcriptase, or M-MLV reverse transcriptase RNase H minus or any other modifications thereof. Any modified RNAs obtained in accordance with any or all afore described steps and further outlined in FIGS. 1 to 3 can be used in a cDNA synthesis reaction in which a DNA copy of an RNA template is synthesized by means of a reverse transcriptase. This reaction requires primers that can hybridize to the RNA template and initiate DNA synthesis. In one example, a set of primers is used having a random sequence at their 3′-end followed by a defined sequence. A region of defined sequence may be useful for the later manipulation of a DNA, but it is not required for the priming of the reverse transcription reaction. Hence, the invention can make use of primers having random sequences only. Such a random sequence on its own or as part of an oligonucleotide having also defined regions can be 4 to 6 nucleotides in length, 6 to 10 nucleotides in length, or 10 to 15 nucleotides in length. The use of random primers leads to the synthesis of DNA fragments having sequences complementary to sequences at the 5′-end of the RNA template. Since a random primer can hybridize to any region within the RNA template, the reaction will give raise to a mixture of DNA molecules of different length. Although random priming does not allow for the preparation of a full-length cDNA, it may have advantages in reaching the true 5′-ends of long RNAs, which are otherwise difficult to obtain due to the limitations of the reverse transcriptase reaction. In a different example, an oligo-dT primer is used to hybridize to the polyA tail commonly found at the 3′-end of many mRNA species. An oligo-dT primer can be applied to primer DNA synthesis from any modified RNA template comprising a polyA tail. In contrast to random priming, oligo-dT priming is commonly used for the synthesis of a full-length cDNA that reflects the entire sequence of an RNA molecule. Moreover, primers of defined sequence may be used to prime the reverse transcriptase reaction as, for example, commonly used for applications such as the RACE (Rapid Amplification of cDNA Ends) method. Other methods for priming full-length cDNA synthesis are disclosed in WO2006003721, hereby incorporated herein by reference. Hence, the invention provides different methods for the preparation of a DNA transcript from a modified RNA as further outlined in FIGS. 4 and 5.

The aforementioned synthesis of a DNA from an RNA template leads to the formation of a double-stranded DNA/RNA molecule. The RNA portion within any such double-stranded DNA/RNA molecule can be removed by means of an RNA degrading enzyme or changes in the pH of the reaction buffer. For example, the enzyme RNase H specifically digests RNAs within double-stranded DNA/RNA molecules, making it a preferable enzyme to practice the invention. The removal of RNA applies for any kind of cDNA regardless of the priming, either random priming, specific priming or oligo-dT priming, used for the reverse transcription reaction. Examples for the removal of RNA from a DNA/RNA template are described in Sambrook J. and Russuell D. W., Molecular Cloning, A Laboratory Manual, Cold Spring Harbor Laboratory Press, New York, 2001, hereby incorporated herein by reference. Any such treatment of a double-stranded DNA/RNA molecule releases the DNA strand which can be obtained as a single-stranded DNA molecule whose entire template or most of the template had been made of ribonucleotides. In case the DNA/RNA hybrid contains regions of double-stranded DNA, for example, when parts or the entirety of the oligonucleotide added to the RNA molecule at a previous step have been made out of DNAs, the removal of the RNA portion of the hybrid molecule will lead to the preparation of a DNA molecule comprising regions of a double-stranded DNA at the end equal to the 5′-end of the RNA template. Hence, the invention provides a method for preparing single-stranded and/or partly single-stranded DNA molecules comprising sequence information derived from an RNA molecule which may be an mRNA or a total RNA or sequence information introduced by means of manipulation of such an RNA molecule.

Single-stranded DNA molecules are important for DNA analysis and manipulation, and many applications and technologies in molecular biology and biotechnology require the strand-specific preparation of single-stranded DNA. Such applications include, but are not limited to, the preparation of a template DNA for sequencing or for strand-specific DNA synthesis including synthesis of labeled probes, the replacement of thymine residues by uracil, the introduction of point mutations, the preparation of testers and drivers for subtractive hybridizations or the detection and isolation of individual clones in a mixture of various DNA or RNA molecules, the detection and analysis of single nucleotide polymorphisms (SNPs), and the preparation of microarrays. Those methods and their applications are well known to those skilled in the art of molecular biology and are further described by Sambrook J. and Russuell D. W., Molecular Cloning, A Laboratory Manual, Cold Spring Harbor Laboratory Press, New York, 2001, hereby incorporated herein by reference.

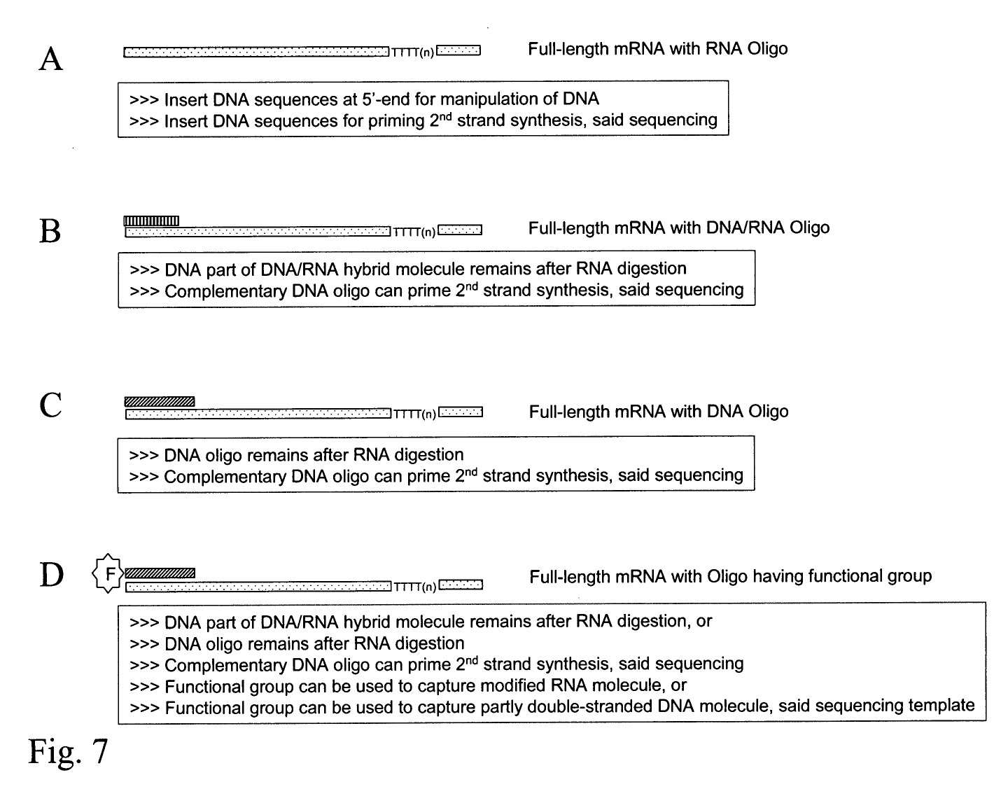

DNA templates obtained by means of the invention may be distinct in their features depending on the nature of the oligonucleotide added to the RNA species at the early stage as, for example, shown in FIGS. 2 and 3 and outlined in more detail in the forgoing. Hence the invention can be used for the preparation of single-stranded or partly single-stranded DNA molecules such as those depicted in FIG. 7. For example, if an RNA oligonucleotide has been attached to an RNA, and the resulting modified RNA molecule has proceeded to the cDNA synthesis by means of a reverse transcriptase, the removal of the RNA portion from the RNA/DNA hybrid molecule will lead to a single-stranded DNA molecule having sequences complementary to the RNA template, sequences complementary to the RNA oligonucleotide added to the RNA at an early stage at the 3′-end, and sequences directly derived from the primer used in the reverse transcription reaction at the 5′-end (compare FIG. 7A). Thus, such a DNA molecule comprises a region of potentially unknown sequence in the center flanked by regions of known sequence which are derived directly or indirectly from an oligonucleotide attached to RNA. Since the sequences of the flanking regions are known, such a molecule can be used as a direct template for sequencing and manipulation. For example, a primer having complementary sequence to sequences at the 3′-end of the DNA molecule can be used to prime a sequence reaction. A classical approach for sequencing a DNA template, for example, by the Sanger method is described in Sambrook J. and Russuell D. W., Molecular Cloning, A Laboratory Manual, Cold Spring Harbor Laboratory Press, New York, 2001, hereby incorporated herein by reference. However, the invention is not limited to the sequencing of the DNA template by the Sanger method, and recently a number of alternative sequencing methods have been developed as further outlined in Metzker M. L. Genome Res. 15, 1767-1776 (2005), Kling J., Nature Biotechnology 23, 1333-1335 (2005); and Shendure J. et al., Nature Review Genetics 5, 335-344 (2004), both of which are hereby incorporated herein by reference. Some of those approaches are subject to commercial applications as offered by companies like 454 Life Sciences, found at http://www.454.com/, Helicos BioScience, at http://www.helicosbio.com/, Solexa, at http://www.solexa.com/Company/overview.htm, Visigen, at http://www.visigenbio.com/index.html, or GeneoVoxx GmbH, at http://www.genovoxx.de/ (the information found in any or all of those web pages is hereby incorporate herein by reference). Since the invention provides a method for designing the opposite ends of a DNA template, the DNA template can be designed in such a way that the molecule has the necessary features, for example, as needed for the sequencing by any of the aforementioned methods or new developments within the field. For example, for sequencing by the method offered by 454 Life Sciences specific primer sites at the opposite ends of the sequencing template are required to allow for a clonal amplification of each DNA molecule by emulsion PCR. In a preferred example, the DNA template contains two different sequences at its 5′- and 3′-end that are suitable for single-molecule emulsion PCR amplification as described in Margulies M. et al., Nature 437, 376-80 (2005), hereby incorporated herein by reference. In a different example, an DNA/RNA hybrid oligonucleotide has been attached to an mRNA, and the resulting modified RNA molecule has been forwarded the cDNA synthesis by means of a reverse transcriptase, the removal of the RNA portion from the RNA/DNA hybrid molecule will lead to a partly single-stranded DNA molecule having a region of double-stranded DNA at the 3′-end of the longer DNA strand corresponding to the 5′-end position of the RNA. Further this DNA molecule would comprise sequences complementary to the RNA template, sequences complementary to the RNA/DNA oligonucleotide added to the RNA at an early stage at the 3′-end, and sequences directly derived from the primer used in the reverse transcription reaction at the 5′-end (compare FIG. 7B). In this constellation, the double-stranded region of the DNA molecule contains a short stretch of DNA which can function as a primer for a DNA polymerase reaction. Hence, the invention provides a method for directly preparing a sequencing template that does not require any further addition of a sequencing primer. In just a different example, an DNA oligonucleotide has been attached to an mRNA, and the resulting modified RNA molecule has been forwarded to cDNA synthesis by means of a reverse transcriptase, the removal of the RNA portion from the RNA/DNA hybrid molecule will lead to a partly single-stranded DNA molecule having a region of double-stranded DNA at the 3′-end of the longer DNA strand corresponding to the 5′-end position of the RNA. Further this DNA molecule would comprise sequences complementary to the RNA template, sequences complementary to the DNA oligonucleotide added to the RNA at an early stage at the 3′-end, and sequences directly derived from the primer used in the reverse transcription reaction at the 5′-end (compare FIG. 7C). The template obtained by these methods is largely similar to the template described in the FIG. 7B, besides that the double-stranded region would stop exactly at the 5′-end of the original RNA template. Hence, by the use of a DNA oligonucleotide or an DNA/RNA hybrid oligonucleotide the experiment can be designed in such a way that the primer or its priming site is in direct proximity to the 5′-end of the original RNA or separated from the 5′-end of the original RNA by 1 to 5 nucleotides, 5 to 10 nucleotides, 10 to 15 nucleotides, or more than 15 nucleotides. A DNA molecule as shown in FIG. 7C can otherwise be forwarded to a sequencing reaction in the same manner as already outlined in the above for the template depicted in FIG. 7B. In a modification of the aforementioned examples that led to the preparation of the DNA molecules depicted in FIGS. 7A and 7B, the DNA/RNA oligonucleotide or the DNA oligonucleotide attached to the RNA contains a function group. In this example, the invention would lead to the preparation of a partly single-stranded DNA molecule having a region of double-stranded DNA at the 3′-end of the longer DNA strand corresponding to the 5′-end position of the RNA, where the shorter fragment within the double-stranded regions contains a functional group. The longer DNA strand within the molecule would comprise sequences complementary to the RNA template, sequences complementary to the DNA or DNA/RNA oligonucleotide added to the RNA at an early stage at the 3′-end, and sequences directly derived from the primer used in the reverse transcription reaction at the 5′-end (compare FIG. 7D). Besides the use of such DNA template in sequencing reactions, the functional group would allow for a direct attachment of the sequencing template to a support or a matrix. The functional group and any molecule attached to such a functional group can bind to a binding molecule attached to the matrix. Such matrix can be selected depending on the nature of the functional group. For example, if the applied functional group is a reactive group such as an amino group, then in a chemical reaction the reactive group can be used to form a covalent bound to the matrix. When the functional group is biotin, then the related matrix may be avidin or streptavidin. When the functional group is digoxigenin, the matrix may be a digoxigenin-binding molecule (see Roche Diagnostics GmbH Catalog, which is hereby incorporated herein by reference). When the functional group is an oligonucleotide or an Identifier Sequence, the matrix may be a nucleic acid molecule having a complementary sequence, or when the functional group is an antigen, the matrix may be an antibody or an antibody-binding protein such as protein I or protein G. Hence the invention provides a method for preparing DNA molecules or sequencing templates that contain a primer site and have features for direct binding to a support or matrix. In a preferable example of the invention, such templates would be directly applied to new sequencing methods, where individual molecules are bound to a support for direct sequencing. Such sequencing methods would include, but are not limited to, those offered by 454 Life Sciences at http://www.454.com/ or under development by Helicos BioScience at http://www.helicosbio.com/, Solexa at http://www.solexa.com/Company/overview.htm, Visigen at http://www.visigenbio.com/index.html, or GeneoVoxx GmbH at http://www.genovoxx.de/(information found at all and any these web pages is hereby incorporate herein by reference).

In the aforementioned examples, the invention provided a method for modifying 5′-ends of mRNAs. However, the invention is not limited to the modification of the 5′-ends. In accordance with the examples given above for the priming of cDNA synthesis in reverse transcription reaction and depicted further in FIGS. 4 and 5, a common primer such as an oligo-dT primer, or a set of primers such as random primers or primers having specific sequences are used to prime the first-strand cDNA synthesis from an RNA template. Primers for the priming of cDNA synthesis may comprise single-stranded DNA, or are partly single-stranded and partly double-stranded DNA such as those disclosed in WO2006003721, hereby incorporated herein by reference. A primer may comprise a region having a sequence complimentary to sequence information within the RNA template, and may comprise other sequence information designed for later use in the manipulation of the cDNA or function as an Identifier Sequence. The nucleotides that hybridize to the RNA may have a random sequence, may be taken from a public database to achieve priming of a specific RNA species, or may be composed of a longer stretch of dT nucleotides to hybridize to the polyA tail of an mRNA. Information on the sequence of an RNA molecule can be obtained by searches in a public database known to a person skilled in the art such as NCBI at http://www.ncbi.nlm.nih.gov/, EMBL at http://www.ebi.ac.uk/Databases/, or DDBJ at http://www.ddbj.nig.acjp/, and in many cases the obtained information will be sufficient to design 3′-end specific primers. In a different example, such sequence information may also be used to design primers that target for defined regions within RNAs, such as a splice site or one of the ends of a coding region called an open reading frame. The primer used to prime the reverse transcription reaction may comprise only sequences complementary to RNA. In this example, the resulting cDNA obtained from such a reaction would have sequences at its 5′-end complementary to sequences within the RNA. In a different example, the primer would comprise a sequence complementary to a sequence within RNA and sequence information unrelated to sequence information from RNA. Hence, the invention provides a method for introducing new sequence information into a cDNA molecule so that the sequence information extends the cDNA at its 5′-end. Such additional sequence information can be used for the manipulation of the cDNA. A person skilled in the art knows many enzymatic activities that depend on binding to specific sequences or recognition sites. Many such enzymes can be commercially obtained from different suppliers including, but not limited to, FERMENTAS UAB (Vilnius, Lithuania), New England Biolabs Inc. (Beverly, USA), Promega (Madison, USA), Takara (Tokyo, Japan), Roche (Mannheim, Germany), and GE Biosciences (Cardiff, United Kingdom). Commonly, restriction endonucleases cut only double-stranded DNAs, but do not cut single-stranded DNAs. They therefore could only be applied after a second strand has been synthesized, and may be used, for example, for the purpose of cloning. Hence the invention relates to the full-length cloning of nucleic acid molecules, so that the sequence information obtained from DNA fragments according to the invention. In a particular example, a restriction endonuclease can be used to remove polyA/T stretches from cDNAs as, for example, described by Shibata Y et al. in Biotechniques. 31, 1048-1049 (2001), hereby incorporated herein by reference. Approaches for the removal of polyA/T stretches are of particular importance for methods for obtaining sequencing tags from 3′-ends of RNAs including, but not limited to, those disclosed in patent applications US2005/059022, US2005/0255501, WO2004/050918, and U.S. Pat. No. 6,136,537, all of which are hereby incorporated herein by reference.

In another example, the primer used in the reverse transcription reaction includes a functional group attached to such primer. Such functional group will be incorporated into the cDNA regardless of the nature of the primer such as a set of random primers, a specific primer, or an oligo-dT primer. Hence the invention provides a method for the preparation of cDNA fragments having a modified 5′-end with a functional group. A person skilled in the art knows many different functional groups that have an affinity to bind to a binding molecule. A functional group may include, but is not limited to, a reactive group, an amino group, biotin, digoxigenin, an antibody, an antigen, a protein, and a nucleic acid binding molecule. The functional group and any molecule attached to such a functional group can bind to a binding molecule presented on a matrix. For the purpose of the invention a matrix may be selected from any immobilized form of avidin, streptavidin, a digoxigenin-binding molecule, an antibody and its ligand and/or chemical matrix. If the applied functional group is a reactive group such as an amino group, then in a chemical reaction the reactive group can be used to form a covalent bound to the matrix. When the functional group is biotin, then the related matrix is avidin or streptavidin. Similarly, when the functional group is digoxigenin, the matrix is a digoxigenin-binding molecule (see Roche Diagnostics GmbH Catalog, which is hereby incorporated herein by reference). When the functional group is an antigen, the matrix may be an antibody or an antibody-binding protein such as protein I or protein G. Modified oligonucleotides can be commercially obtained from many providers. Frequently, biotin-labeled oligonucleotides are used in the field. As an example for the preparation of different modified oligonucleotides see the web pages of MWG Biotech at http://www.mwg-biotech.com/html/s_synthetic_acids/s_modifications.shtml, Invitrogen at http://www.invitrogen.com/content.cfm?pageid=9900, or Operon at http://www.operon.com/, the information found in those pages is hereby incorporated herein by reference. In a different example, the oligonucleotide may have recognition sites for a DNA binding protein. Many DNA binding proteins are known to a person skilled in the art, and they can be of natural occurrence or may have been prepared by means of protein design. Such DNA binding proteins include, but are not limited to, transcription factors, proteins of regulatory function that bind directly or indirectly to recognition sites in genomic DNA. Every living organism contains a large number of transcription factors. As an example, Kanamori M. et al. have published a database on all the known transcription factors from mouse in Biochem Biophys Res Commun, 322, 787-93 (2004), hereby incorporated herein by reference. Transcription factors are distinct by their affinity to different recognition sites in such a way that transcription factors bind to specific sequences. This specificity can be used for the enrichment of DNA molecules comprising recognition sites for a given transcription factor and/or group of transcription factors. However, the binding specificity of a transcription factor is not limited to binding a certain sequence, as a person skilled in the art knows proteins that rather recognize structures than specific sequences. For example, the transcription factor DAX-1 can bind to different DNA structures such as those described by Zazopoulos E. et al., Nature 390, 311-315 (1997), hereby incorporated herein by reference. In a different example, a DNA binding protein may bind specifically to a single-stranded DNA. Single-stranded-DNA binding proteins including, but not limited to, SSB from E. coli, the product of the phage T4 Gene 32, the adenovirus DBP, an antibody directed against a single-stranded DNA, calf thymus UPI, or any mixture thereof. In a different example, the binding protein may be MutS or a member of the MutS gene family. In a further different example, the oligonucleotide may have regions of a given sequence that can be used as an Identifier Sequence. Such a given sequence or the Identifier Sequence can be used to mark the origin of a sample, or can function as a tag to specifically capture a modified RNA or any DNA derived thereof by means of hybridization to a nucleic acid molecule or the like having a sequence complementary to the Identifier Sequence, or can be used as a specific and selective priming site for any of the aforementioned enzymatic reactions. As such, the Identifier Sequence can be a selective priming site for the second-strand synthesis by a DNA polymerase, the amplification, for example, by means of a PCR reaction, the preparation of a single-stranded DNA, or the priming of a sequence reaction. Hence, in combination with the aforementioned methods for introducing different oligonucleotides into a cDNA which is derived from RNA molecules among a plurality of RNAs.

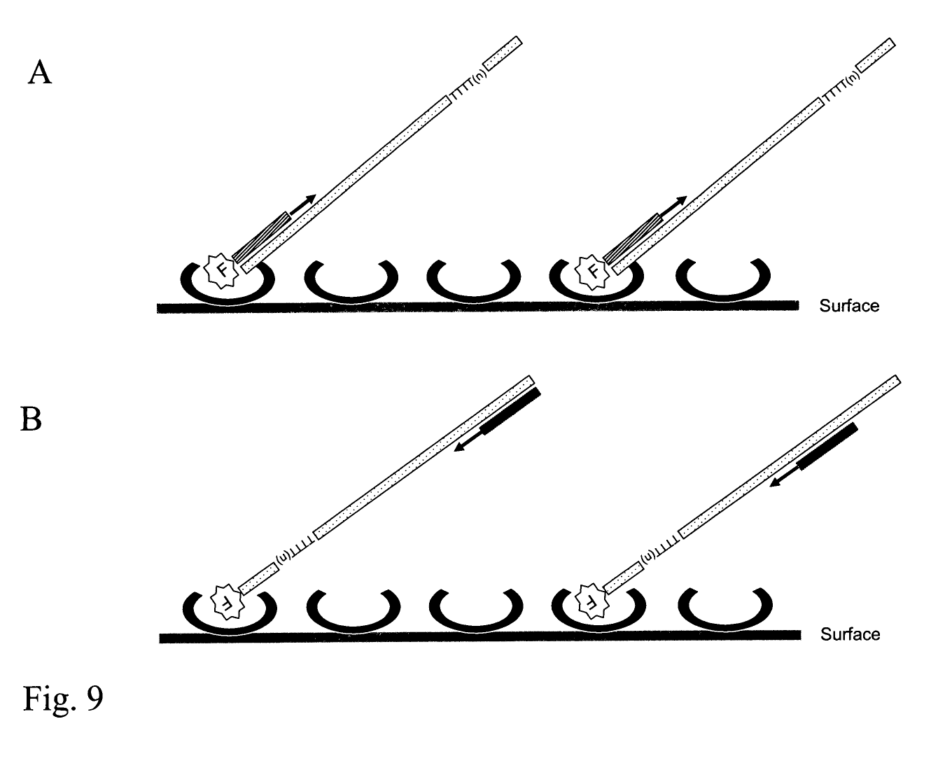

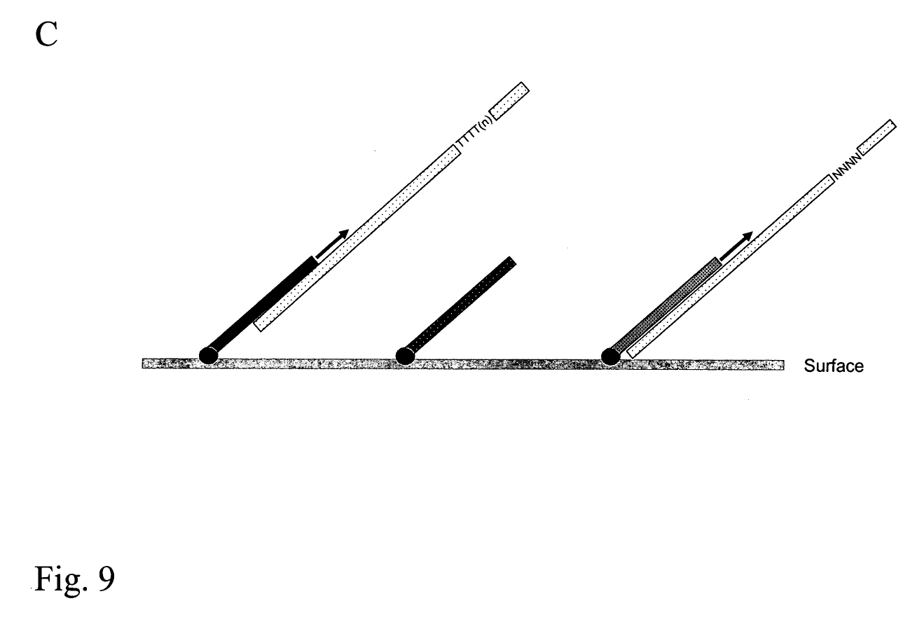

In accordance with any of the steps outlined in the forgoing, the invention provides a method for introducing a functional group at a position equal to the 5′-end of an RNA. In addition, the invention provides a method for introducing a functional group at a position equal to the 3′-end of RNA or the 5′-end of a first strand cDNA. Hence, the invention provides a method for introducing a functional group at either end of a cDNA derived from an RNA. The functional group attached to an RNA or a cDNA has a binding affinity to another molecule, and the functional group can be used to capture the modified RNA or cDNA and attach molecules to a surface. Examples for combinations of a functional group and a binding molecule may include, but are not limited to, a reactive group such as an amino group that can be used to form a covalent bound to the matrix in a chemical reaction the reactive group, biotin binding to avidin or streptavidin, digoxigenin binding to a digoxigenin-binding molecule, an oligonucleotide binding to a complementary sequence, an antigen binding to an antibody, or an antibody binding to an antibody-binding protein such as protein I or protein G. Depending on the location of the functional group the modified RNA or DNA may be attached to a surface in a different manner or orientation. For example, a partly double-stranded cDNA molecule can be attached to a surface by means of an interaction of the functional group at a position equal to the 5′-end of RNA. In this example, the partly double-stranded region of the cDNA molecule enables the sequencing of the cDNA fragments from the end equal to the 5′-end of RNA (compare FIG. 9A). In a different example, a single-stranded cDNA molecule is attached to a surface by means of an interaction of the functional group attached to the 5′-end of a single-stranded DNA molecule which corresponds to the 3′-end of RNA with the surface. While the cDNA is attached to the surface at a position corresponding to the 3′-end of RNA, external primers may be used to determine the position from which the cDNA molecule is sequenced (compare FIG. 9B). In a different example, sequences attached to the modified RNA or the first-strand cDNA may have a sequence complementary to oligonucleotide presented on a surface. FIG. 9C depicts the principle of having a single-stranded RNA or cDNA molecule attached to a surface by means of hybridization to an oligonucleotide which functions as a primer and which is bound to the surface. The primer on the surface can determine which RNA or cDNA fragment can be attached to the surface, depending on whether or not having a sequence complementary to any of the primers on the surface. Moreover, primers on the surface determine the position from which the cDNA molecule is sequenced. Examples for sequencing on a solid phase have been published by Stahl, S. et al., Nucleic Acid Research 16, 3025-3038 (1988) or Lindroos K. et al., Nucleic Acid Research 29, No. 13 e69 (2001), both of which are hereby incorporated herein by reference. A person skilled in the art knows of different approaches to synthesize oligonucleotides of defined sequence directly on a support, or know different methods for binding such oligonucleotides onto a support. Such approaches are commonly used in the preparation of microarrays as further described in Jordan B., DNA Microarrays: Gene Expression Applications, Springer-Verlag, Berlin Heidelberg New York, 2001: Schena A, DNA Microarrays, A Practical Approach, Oxford University Press, Oxford 1999, both of which are hereby incorporated herein by reference. Hence, the invention provides a method for preparing modified nucleic acid molecules and their capturing for analysis such as RNA and DNA sequencing.

In the aforementioned embodiment, the invention provides a method for preparing single-stranded or partly single-stranded RNA and/or DNA molecules. Using a functional group, such molecules can be attached to a surface. Molecules on a surface can be washed by different buffers for purification and further manipulation. Hence, the invention provides a method for purifying single-stranded DNAs, partly single-stranded DNAs, or RNAs. Such a method for purifying of single-stranded DNAs, partly single-stranded DNAs, or RNAs are mandatory for the detection of single molecules as achieved by new technologies including, but not limited to, those described in Metzker M. L. Genome Res. 15, 1767-1776 (2005), Kling J., Nature Biotechnology 23, 1333-1335 (2005) and Shendure J. et al., Nature Review Genetics 5, 335-344 (2004), both of which are hereby incorporated herein by reference. Hence, the invention relates to the use of single-stranded DNA, partly single-stranded DNA, or RNA molecules for directly obtaining sequence information thereof. In a preferable embodiment, the invention relates to obtaining sequence information from defined regions of single-stranded DNA fragments. In a preferable example, the 5′-end specific sequence information of RNA is obtained from a DNA fragment prepared according to the invention having sequence complementary to the 5′-end sequence of an RNA molecule. Thus, the invention relates to obtaining sequence information from RNAs.

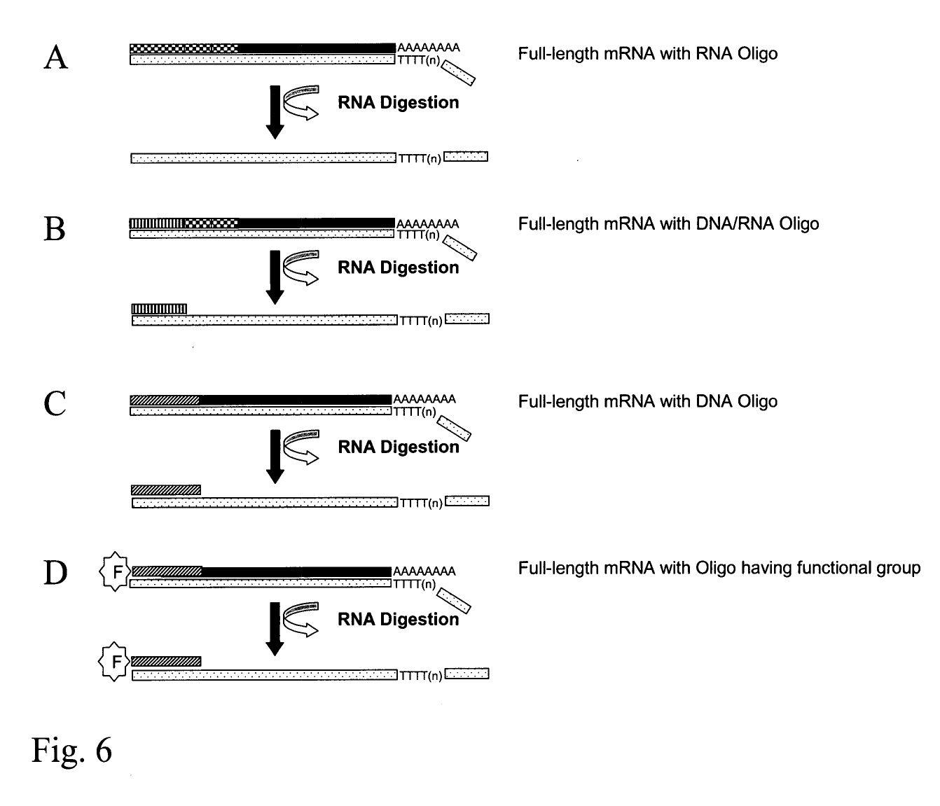

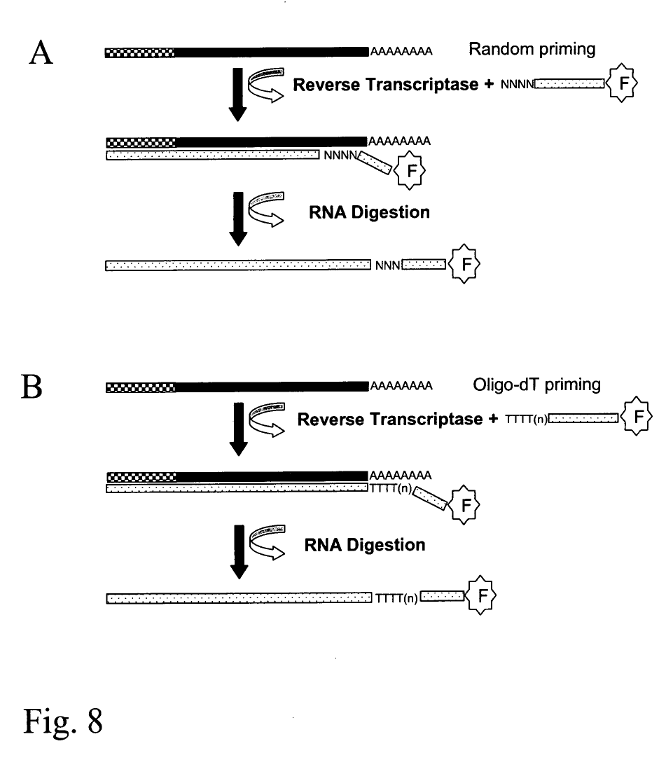

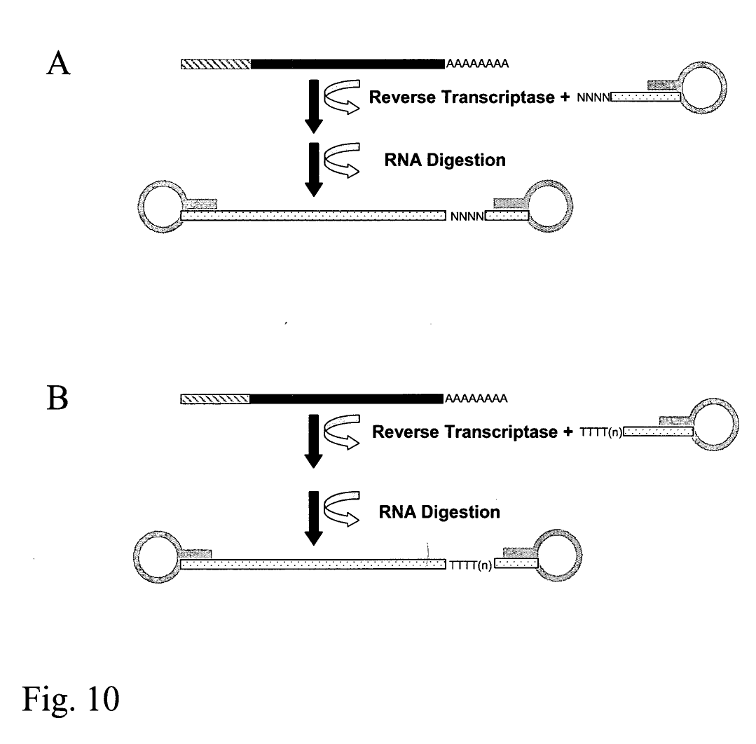

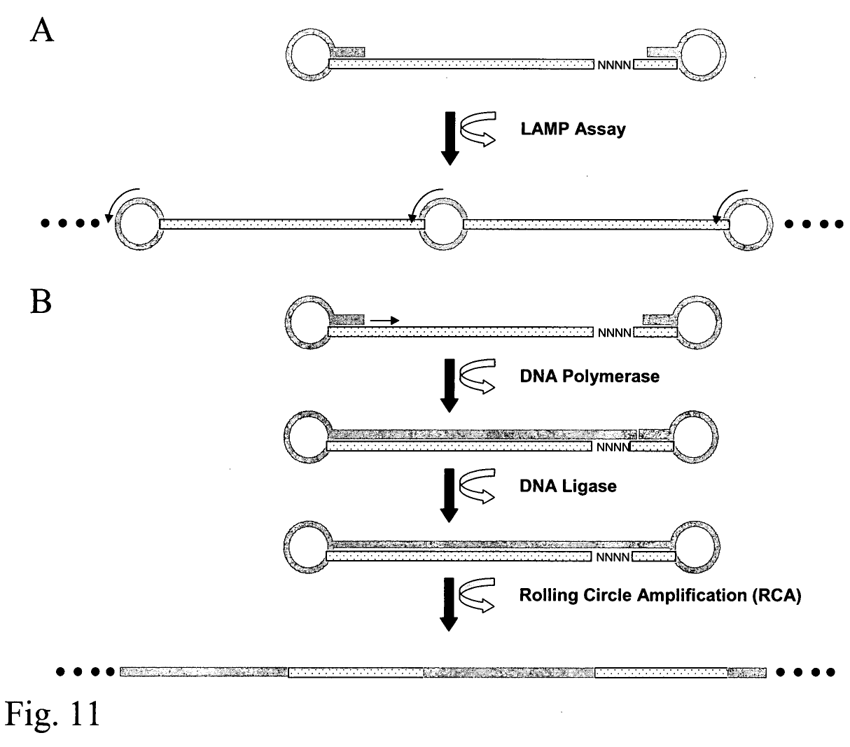

In accordance with the forgoing and any of the steps outlined in FIGS. 1 to 7, the invention provides a method for introducing an oligonucleotide at a position corresponding to the 5′-end of RNA. In addition, in accordance with the foregoing and any of the steps outlined in FIG. 8, the invention provides a method for introducing an oligonucleotide at a position equal to the 3′-end of RNA. Hence, the invention provides a method for introducing specific sequences at either end of a cDNA derived from an RNA and a method for preparing cDNA fragments having modified ends. In one example, the sequences introduced by means of the invention have the ability to form hairpin structures in which singe-stranded DNA molecules that fold into such a configuration that complimentary sequences within the single-stranded DNA molecule form a region of double-stranded DNA with a closed loop at one end. Thus, following the aforementioned procedures, a cDNA molecule can be obtained from an RNA that is modified in such a way that it has hairpin structures at the opposite ends (compare FIG. 10). Depending on whether the first-strand cDNA synthesis has been primed by a set of random primers, a specific primer, or an oligo-dT primer, such a molecule may comprise partial sequences derived from an RNA or the entire sequence derived from an RNA (a full-length cDNA). In one example, a single-stranded cDNA molecule prepared in accordance with the foregoing and having a hairpin structure at the end equivalent to the 5′-end of RNA can be directly sequenced, in which the hairpin structure will function as the priming site for the sequencing reaction. In a different example, a single-stranded cDNA molecule prepared in accordance with the foregoing and having a hairpin structure at both ends can be amplified by making use of the two hairpin structures. As depicted in FIG. 11A, a single-stranded DNA fragment having a loop structure at each end is a template for Loop-Mediated Isothermal Amplification (the so-called LAMP method) disclosed in Notomi T. et al., Nuc Acids Res. 8, e63 (2000), hereby incorporated herein by reference. Hence, a DNA molecule prepared according to the invention can be amplified in such a way that a polymer of repetitive sequences is obtained, and the loop structures within such a polymer can be used to prime the extension of the amplification reaction or can be used to drive a sequence reaction. In one example, the amplified fragment is 25 to 50 bp long, or 50 to 100 bp long, 100 to 200 bp long, or 200 to 300 bp long. In a different example, the amplified DNA fragment is over 300 bp long. Hence, a DNA molecule prepared according to the invention can be amplified in such a way that a linear polymer of repetitive sequences is obtained, and such a polymer contains sequences that can be used to drive a sequence reaction.

In a preferable example, a DNA molecule having loop structures at each end is converted into a circular single-stranded DNA molecule by steps comprising a first reaction in which the free 3′-end of one hairpin structure is extended by means of a DNA polymerase lacking any exonuclease and strand-displacement activities. Such DNA polymerases include, but are not limited to, any reverse transcriptase such as the M-MuLV Reverse Transcriptase, H Minus M-MuLV Reverse Transcriptase, Superscript II, Superscript III, AMV Reverse Transcriptase, MonsterScript, Expand Reverse Transcriptase, or any mixture thereof. Other DNA polymerases may include, but are not limited to, the Klenow fragment of DNA polymerase I, T4 and T7 DNA polymerases, DNA polymerase I, Taq polymerase, Tfl DNA polymerase, Tth DNA polymerase, Tli DNA polymerase, or any other DNA polymerase known in the field. Due to the lack of a strand displacement activity the DNA polymerase will stop when reaching the 5′-end of the opposite hairpin structure. In a second reaction step, the open ends of the single-stranded DNA molecule are ligated to each other to form a circular DNA molecule. Such a ligation reaction can be performed by any DNA ligase including but not limited to the T4 DNA ligase, E. coli DNA ligase, or Taq DNA ligase. Circular single-stranded DNA molecules can be amplified by means of the rolling circle amplification method (so-called RCA method). The RCA reaction is driven by a DNA polymerases that can extend oligonucleotide primers on a circular template in an isothermal reaction as further describe in U.S. Pat. Nos. 5,854,033 and 6,143,495, both of which are hereby incorporated herein by reference. The reaction product is a linear chain of single-stranded DNA which contains copies of a template linked in tandem. Depending on the reaction conditions and time, the reaction product may contain tens, hundreds, or even thousands of copies of the original template in one molecule. Special DNA polymerases for use in RCA reactions are known to a person skilled in the art in the field including, but not limited to, the phi29 DNA Polymerase, which has a strong strand displacement activity needed for efficient isothermal DNA amplification. A person skilled in the art in the field knows many different applications and modifications of the RCA method. For further reference on the RCA method, see the following review articles: Gusev, Y. et al., American J. Pathology 159, 63-69 (2001), or Zhang D. et al. Clin. Chim. Acta. 363, 61-70 (2006), both of which are hereby incorporated herein by reference. Hence, a DNA molecule prepared according to the invention can be amplified in such a way that a linear polymer of repetitive sequences is obtained, and such a polymer contains sequences that can be used to drive a sequence reaction.

In one example, the RCA reaction is performed in such a way that the reaction product is directly or indirectly bound to a defined location or a point called the point of detection, analysis, or sequencing. As one example, Nallur G. et al., Nucleic Acid Res, 29, el 18 (2001) describes procedures for RCA mediated signal amplification on glass slides. In this example, RCA is the enabling step to perform clonal amplification of individual targets within a plurality of nucleic acid molecules, in which each molecule is amplified at a defined location on a surface. Here, the RCA reaction can be performed in a highly parallel manner without taking the risk of amplification biases known, for example, from classical PCR reactions. Using primers that are attached to a surface in the RCA reaction, an arrayed matrix of reaction products can be obtained so that each reaction product contains multiple copies of the template in one molecule. Hence, the RCA reaction can greatly amplify the sensitivity of detection or analysis, or can make it possible to perform a reliable sequencing reaction at a given location.

In a different example, the RCA reaction is used to prepare a template for detection, analysis, and/or sequencing at a defined location, where the template is subject to one or more detection steps, analysis, or sequencing reactions. Hence, the invention provides a method of sequencing a template in a first step, removing the amplification products produced during the sequence reaction from the sequencing template in a second reaction step, and re-sequencing the same template in a third reaction step by a different primer at the same location. Such a course of reactions may be performed to obtain two different sequencing reads from one template, three different sequencing reads from one template, four different sequencing reads from one template, five different sequencing reads from one template, or even more than five different sequencing reads from one template. The covalent attachment of DNA to a surface is discussed and it is shown that the covalent bound allows for at least 30 cycles of hybridization and stripping of the hybridized DNA in Beier M. and Hoheisel J. D., Nucleic Acid Research, 27, 1970-1977 (1999). Hence, the invention provides a method for providing more than one type of sequence information from a template, in which different sequencing reactions are performed at the same location, at which the link is defined between the different sequencing reads obtained from the same template.

In another example, the RCA reaction is performed to prepare a reaction product that contains multiple copies of the sense and anti-sense strands of an original RNA molecule. Such reaction products are obtained when a circular template for the reaction is prepared in accordance with the steps shown in FIG. 11B so that the circular template contains the sense and antisense strands found within a double-stranded cDNA obtained from an RNA and is connected by hairpin structures at the positions corresponding to the opposite ends of the original RNA. This template is bi-directional. In a preferable example of the invention, the hairpin structures contain priming sites that enable the sequencing of the sense and antisense strands from their ends. Hence the invention provides a method for obtaining end-sequences from the opposite ends of RNA molecules, where the RNA molecule is converted into a cDNA, the cDNA is made double-stranded to have a sense and an antisense strand having the sequence of the initial RNA molecule, the sense and antisense strands within the double-stranded cDNA are connected by hairpin structures to form a circular molecule comprised of single-stranded DNA, the circular single-stranded DNA molecule is amplified by means of an RCA to produce a linear DNA template for sequencing comprising the sense and antisense strands, and sequence information from the opposite ends of the DNA or the original RNA is obtained in two or more consecutive sequencing reactions performed at the same location. In this example, the invention can be applied to determine the end sequences of an RNA, the boarders of transcripts, locations of transcriptional initiation and termination within the genome, or the end-sequences of any DNA molecule. In a different example, the invention can be applied to determine the end-sequences of defined regions within an RNA, a cDNA, or genomic DNA. The borders of such defined regions may be defined by specific steps during their preparation. The fragments, may also be of biological origin and they may be produced by entirely random cutting. Moreover, sequencing primers can be designed to hybridize to any region within the template, similar to classical primer walking strategies, or may be directed to specific regions such as splice sites within the template. It is within the scope of the invention to convert any double-stranded DNA into a bi-directional template, for example, by ligating oligonucleotides having hairpin structures to the ends of a linear double-stranded DNA to form a circular single-stranded DNA molecule. The circular single-stranded DNA molecule is amplified by means of an RCA to produce a linear DNA template for sequencing comprising the sense and antisense strands of the original DNA molecule, and sequence information from the opposite ends of the DNA is obtained in two or more consecutive sequencing reactions performed at the same location. In this example, the invention, for example, can be used to determine end-sequences from exons, genomic fragments obtained by chromatin IP, borders for hypersensitive sites, and so on.

In a different embodiment, the invention relates to the use of Identifier Sequences introduced at the 5′-end of RNAs or at regions equivalent to the 3′-end of RNAs, and the use thereof. As outlined in the forgoing, the invention provides a method for introducing specific sequences or Identifier Sequences at the opposite ends of a cDNA as prepare in accordance with the invention. In a preferable example, the Identifier Sequences are located in the close proximity of the ends of the RNA or cDNA to enable there sequencing within the same sequencing reaction used to obtain sequence information from the RNA or cDNA itself. Identifier Sequences may be designed according to certain rules to fulfill their functions which are unique within a given experiment. An Identifier Sequence may be 1 bp long, 2 bp long, 3 bp long, 4 bp long, 5 bp long, 6 bp long, 6 to 10 bp long, 10 to 15 bp long, 15 to 20 bp, or longer than 20 bp. Preferable Identifier Sequences are 6 to 12 bp in length or 25 to 75 bp in length. An Identifier Sequence may be of arbitrary nature: they may have random sequences. They may be designed by computational means, taken from a biological sample or artificially created. They may also comprise recognition sites for restriction endonucleases or other enzymes and proteins, or priming sites. Identifier Sequences can be designed in accordance with any or all for the following rules:
They should have sufficient length to enable identification by means of sequencing or hybridization.
The sequences of different Identifier Sequences used within the same experiment should be distinct.
Different Identifier Sequences used within the same experiment should be distinct at more than one position to enable a clear identification even if sequencing errors occur within the Identifier Sequence.
Identifier Sequences should avoid sequences having structure or sequences that may interfere with the sequencing reaction (e.g. G-rich sequences, or palindromes).
Identifier Sequences may be selected to form stable hybrids with complementary sequences.
Identifier Sequences may have sequences that enable specific manipulations or binding to dedicated proteins, e.g. restriction endonucleases or transcription factors.
Identifier Sequences should avoid sequences that may interfere with the manipulation of RNA and DNA while performing the invention (e.g. they should not have recognition sites for restriction endonuleases used during the manipulation process).

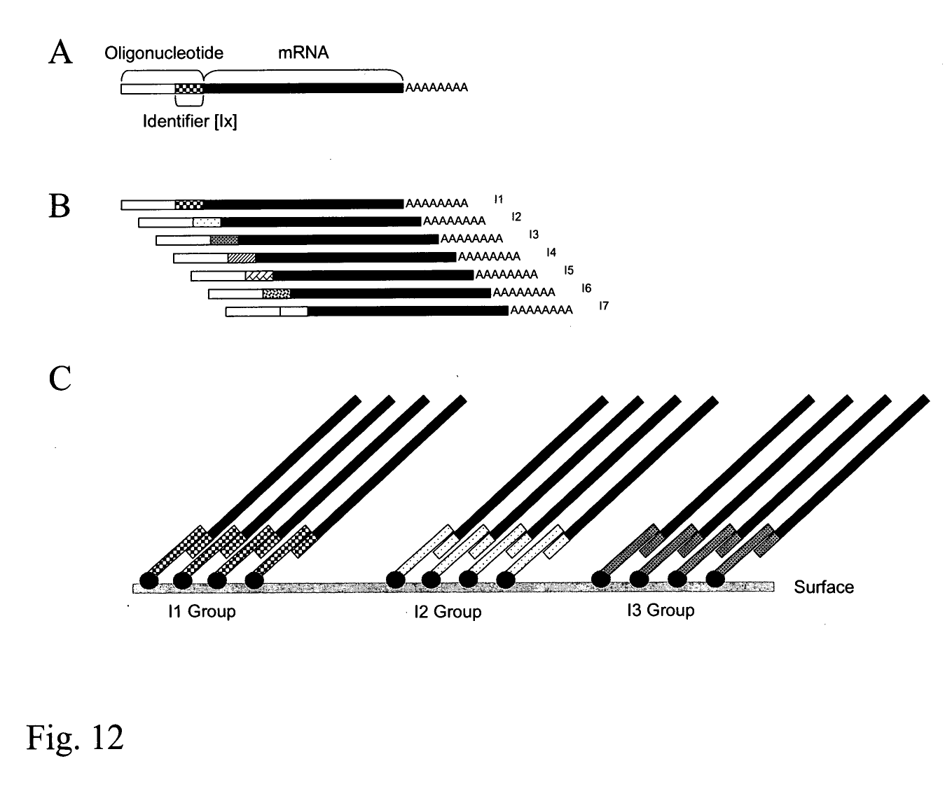

In a preferable example of the invention, Identifier Sequences are used to mark the origin of a sample within a pool of samples, in which all members of the pooled sample are manipulated jointly in the same experiment. The samples within the pooled samples should be mixed as early as possible, preferably already as modified RNA samples. A sample obtained by mixing different RNA samples having different Identifier Sequences would create a “pooled sample” comprising different forms of modified RNAs (compare FIG. 12, Panel B). Therefore the Identifier Sequences are preferably located near the 5′-end of the RNA, and as such, they are introduced during the initial steps for the modification of mRNA or total RNA molecules.

In one embodiment, the Identifier Sequences are used to mark nucleic acid molecules in a particular RNA from multiple biological samples which may include cells from different organisms, tissues or various temporal or treatment stages of a biological experiment, or of different cell types. The pooling of samples within an experimental design may serve different functions including, but not limited to, increasing the complexity of the sample to make full use of the very high throughput of novel sequencing approaches, simplifying the handling of many samples by reducing the number of samples to be handled at the same time, or enabling certain forms of data analysis. In one preferable embodiment, samples are pooled so as to have the same systematic errors over all steps of manipulation for a common statistical analysis as compared to individual experiments in which distinct systematic errors would occur for different samples. For example, in one typical application, the Identifier Sequences are added in proximity of the 5′-end of the RNAs while creating a modified RNA according to the invention, and the modified RNA samples are then mixed prior to the preparation cDNAs thereof. The pooled sample is prepared to have a mixture of different species of modified RNA samples having distinct Identifier Sequences. This pool of modified RNA samples is then treated as a single sample according to the invention in order to obtain data related to the pooled sample. For example, sequencing reads related to the modified RNA samples can be obtained within the pooled library. The sequence information can be determined by any method known in the field, but it is preferable that each sequence read contains the sequence information of the Identifier Sequence plus sequence information derived from the original RNA sample or cDNA. After the determination of the sequencing reads, each individual sequence can be processed computationally in order to recognize the Identifier Sequence, and to group sequence reads having the same Identifier Sequence for further analysis. The sequence information related to the original RNA is analyzed separately from the Identifier Sequences in accordance with the needs of the experimental design. These sequences may relate to so-called “sequence tags” or short sequencing reads comprising partial sequence information derived from an RNA or cDNA. Sequence tags can be used to identify transcripts or certain locations in the genome, or may be used for a statistical analysis on the expression level of transcripts within a pooled sample (for further details on the use of sequencing tags refer to Harbers M. and Carninci P., Nature Meth. 2, 495-502 (2005), hereby incorporated herein by reference). Sequence information may be further stored in databases or by other computational means for the purpose of analysis, archiving, or reference data set building. Such a database could contain, for example, sequence information, the frequency of appearance of each sequence tag within different tissues and cell lines, and annotation data related to transcripts, genes, and functional elements within genomes.

In a different example, the Identifier Sequences are not identified by sequencing, but are used to form specific hybrids with nucleic acid molecules having complementary sequence to the Identifier Sequences bound to a solid matrix or support (compare FIG. 12C). In this example, the Identifier Sequences are used to group samples derived from the same origin to defined locations on a surface. The location will define the nature of the Identifier Sequence or the origin of the RNA, DNA or sample within a pooled sample. Hence, the readout of an Identifier Sequence not necessarily requires direct sequencing, but can be otherwise performed in specific hybridization reactions.

In a different example, the Identifier Sequences are not identified by sequencing or hybridization, but are used to bind specifically to proteins having a binding affinity to the Identifier Sequence that are bound to a solid matrix or support. In this example, the Identifier Sequences are used to group samples derived from the same origin to defined locations on a surface. The location will define the nature of the Identifier Sequence or the origin of the RNA, DNA or sample within a pooled sample. Hence, the readout of an Identifier Sequence not necessarily requires direct sequencing or hybridization, but can be otherwise performed by binding to a protein having high affinity for an Identifier Sequence.

The invention relates further to the sequencing of the regions from DNA fragments obtained according to the invention for the purpose for their annotation by computational means including their statistical analysis, annotation by means of alignments to reference information, and/or mapping to genomic sequences. Thus, the invention relates to a method for gene discovery, gene identification, gene expression profiling, and their annotation.

In another embodiment, the invention relates to the preparation of hybridization probes from the ends of nucleic acid molecules for analyzing such regions by means of in situ hybridization. In a preferred example, the in situ hybridization experiment makes use of a tiling array. In this embodiment, the invention relates further to the design of hybridization probes including, but not limited to, those presented on a microarray.

Thus, the invention provides a method for analyzing nucleic acid molecules and short fragments thereof as needed, for example, for the characterization of biological samples. Moreover, the invention provides a method for fast and effective manipulation of RNA and DNA fragments to make use of such fragments in analytical assays. In this sense, the invention provides a new method for making use of the ever-higher throughput of new sequencing devises and new sequencing technologies.

In another embodiment, modified RNA prepared according to the invention by the use of specific primers in the reverse transcription reaction can be used to determine the real 5′-end sequence similar to protocols known to a person skilled in the art as RACE.

The invention provides a method necessary for obtaining information of value to describe the status of a biological system, namely on the use of genetic information or expression profiles, and the activity of regulatory pass ways or regulatory networks. Hence, the invention relates to the design and performance of analytical assays that can be used in studies in life science and in diagnostic. The invention provides a method for analyzing a biological system and diagnostics.

The invention or parts thereof can be used for the production of a kit containing, among other components, reagents, nucleic acid molecules, and/or enzymes for the manipulation of RNA and the preparation of DNA. In one embodiment, a kit provides the reagents needed to modify RNA. In a different embodiment, a kit provides the reagents used for preparing a DNA template. In a preferable embodiment, a kit provides the reagents to prepare a template for single molecule detection. In a preferable embodiment, a kit provides the reagents for a research purpose. In a more preferable embodiment, a kit provides the reagents for a diagnostic assay.

### EXAMPLES

Key steps of the present invention will now be further explained in more detail with reference to the following examples. All names and abbreviations as used to describe the invention herein shall have the meaning as known to a person skilled in the art.

### Example 1

### Isolation of RNA

To perform an example according to the invention, mRNA or total RNA samples were prepared by standard methods known to a person skilled in the art of molecular biology as, for example, given in more detail in Sambrook J. and Russuell D. W., Molecular Cloning, A Laboratory Manual, Cold Spring Harbor Laboratory Press, New York, 2001, hereby incorporated herein by reference. Furthermore, Carninci P. et al. (Biotechniques 33 (2002) 306-309, hereby incorporated herein by reference) describe a method for obtaining cytoplasmic mRNA fractions. Although the use of cytoplasmic RNA can be preferable, the invention is not limited to this method, and any other approach for the preparation of mRNA or total RNA should allow for the performance of the invention in a similar manner.

The preparation of mRNA from total RNA or cytoplasmic RNA is preferable, but not essential, to perform the invention as the use of total RNA can provide satisfying results in combination with the Cap-selection step performed during full-length cDNA library preparation. The amount of mRNA represents about 1-3% of the total RNA preparations, and it can be subsequently prepared by using commercial kits based on oligo dT-cellulose matrixes. Such commercial kits including, but not limited to, the MACS mRNA isolation kit (Milteny) which provided satisfactory mRNA yields under the recommended conditions when applied for the preparation of mRNA fractions for performing the invention. To perform the invention, one cycle of oligo-dT mRNA selection is sufficient as extensive mRNA purification can cause a loss of long mRNAs.

All RNA samples used to perform the invention were analyzed for their ratios of the OD readings at 230, 260 and 280 nm to monitor the RNA purity. Removal of polysaccharides was considered successful when the 230/260 ratio was lower than 0.5 and an effective removal of proteins was obtained when the 260/280 ratio was higher than 1.8 or around 2.0. The RNA samples were further analyzed by electrophoresis in an agarose gel to prove a good ratio between the 28S and 18S rRNA in total RNA preparations (note rRNA size may change for preparation of total RNA from other species than mammalians), and to show the integrity of the RNA fractions.

### Example 2

### Preparation of a Library of 5′-Derivatized RNA Molecules

This example is a typical protocol for the derivatization of 5′-ends of RNA molecules with RNA oligonucleotides. All reactions were performed in a 500 microliters siliconised microtube and using a siliconized tip each time to avoid nucleic acids losses.

The RNA sample was at first depohosphorylated. The RNA (for instance 1 nanogram to 1 microgram) was added in a tube, together with 2 micrograms of glycogen, in a total volume of 5 microliter. The reaction buffer was 1/10 the common concentration, or 5 mM Bis-Tris-Propane-HCl, 0.1 mM MgCl₂, 0.01 mM ZnCl₂, pH 6.0 at 25° C. Glicogen was used to avoid attachment of RNA to the plastic during the operation. The sample was denatured at 65° C. for 5 minutes to expose the phosphate groups to be later removed, and after being held at 37° C. for 2 minutes the Anctartic phosphatase (New England Biolabs) was added (2.5 units). The sample was treated for 3 hours to overnight at 37° C. Overnight dephosphorylation allowed removal of 98-99% of the phosphate groups. Short incubation could also be performed at 45° C. in the presence of trehalose at 0.6M final, which increased the activity at 45° C.

Then, the Antarctic phosphatase was inactivated at 65° C., but before doing this, the divalent ions had to be chelated. For this reason, 0.55 microliters of a solution of (0.5 M sodium acetate (pH 6.0), 10 mM EDTA, 1% β-mercaptoethanol, and 0.1% Triton X-100) were added. EDTA was chelating the divalent ions and created conditions suitable for the subsequent TAP treatment. The Antarctic phosphatase was also inhibited by EDTA in the buffer. The inactivation was carried out at 65° C. for 5 to 15 minutes.

The forgoing steps were the followed by decapping by simple addition of 0.2 microliters (2 units) of tabacco acid pyrophosphatase (TAP). It was also possible to increase the quantity of the Tap up to 20 units/experiment. The reaction was carried out for 2 hours at 37° C., followed by heat inactivation in this buffer, at 65° C. for 15 minutes, after which the sample was cooled on ice. Optionally, also betain could be added (1 M), which helped melting GC rich secondary structures in RNAs. After this treatment, the TAP did not degrade ATP anymore. ATP was necessary for the subsequent step. Then, the ligation was carried out by adding a “capping RNA” oligonucleotide of any sequence at a concentration of 5 micromolar oligonucleotide. To 6.75 microliters of reaction, 2 microliters of RNA ligase (500 mM HEPES-NaOH (pH 8.0 at 25° C.), 100 mM MgCl₂, 100 mM DTT) were added. DTT inhibited the TAP. Optionally, also hexamino cobaltum chloride (HCC) could be added at 1 mM concentration, but this was optional and not necessary. Polyethylene glicole was then added (PEG 8000) at a final concentration of 25%, ATP at 125 micromolar concentration and finally 10 units of T4 RNA ligase (Fermentas) were added. At such conditions, the resulting mixture of previous buffers was not inhibitory for the ligation steps.

The sample was then ligated for 2 hours to overnight (16 hours) at 20° C. At this point, the former Cap structure of the RNA was replaced with an oligonucleotide, and this could be used for different tests as they appeared in other examples, such as full-length cDNA preparation.

### Example 3

### Activity Testing for Enzymes Used for the Preparation of Modified RNA

The activity of each enzyme used in Example 2 and their buffers were tested by:

(A) Evaluation of the activity of the Antarctic Phosphatase (New England Biolabs). 5′ phosphorylated oligoribonucleotides were dephosophorylated 120 minutes at 37° C. in the following buffers. The oligoribonucleotides were subsequently radiolabelled with T4 Kinase and gamma-³²P-ATP and analysed by PAGE. In absence of prior dephosphorylation, radiolabelling was impossible due to the 5′ phosphate.

(B) Evaluation of the activity of the Tobacco Acid Pyrophosphatase (TAP) (Epicentre). gamma-³²P-ATP was incubated with 2 U TAP in a reaction buffer. The TAP was heated 15 minutes before incubation with radioactive ATP.

(C) For evaluation of the activity of the T4 RNA ligase (Fermentas) A radiolabelled oligoribonucleotide was incubated in presence of an unlabelled oligoribonucleotide. Ligation results in a shift of the electro-mobility in polyacrylamide gel.

### Example 4

### Production of Full-Length cDNA from Modified mRNA

The sample prepared as in the above was desalted using microcon YM-100 filter as described by the manufacturer (Millipore). To the ligated RNA, added were water and reverse transcriptase (RT) primers, which can be obtained by Invitrogen. Used were 800 ng of the primer AGA GAG AGA CCU CGA GCC UAG GUC CGA C for a 20 micro liters reaction, and 3 micro liters of the sorbitol-trehalose mixture (3.3 M stock) were added to have a final concentration of 0.5M Sorbitol and 4% trehalose when making the final RT reaction. The RNA-primer mixture was heated for 10 minutes at 65° C. and then stored on ice while the remaining reagents were prepared. Then a premix composed of 11 micro liters of 2×GC buffer (described in Carninci, Shiraki et al, Biotechniques, 2002; 32, 984-985, hereby incorporated herein by reference) was added, and then 1 micro liter of 10 mM dNTPs stock, and finally, 1 micro liter of MMLV reverse transcriptase (RNaseH minus, Fermentas) were added. The GC buffer system was replaced by a buffer as recommended by the manufacturer. To this reaction mixture, the RNA sample was added, and incubated for 2 min at 25° C. (to anneal the samples), 30 minutes at 42° C., 10 min at 52° C., 10 min at 56° C. before the reaction was stopped. In this way, cDNA was obtained at thigh frequency that spans the 5′-end of the original mRNAs. This was further purified/processed. For instance, it could be treated with proteinase K (addition of 20 micrograms, together with EDTA at 10 mM final concentration, followed by RNA and Proteinase inactivation at 95° C. for 15 minutes. This sample could then be used on C14B (Amersham-Pharmacia) to fractionate the size, or eliminate the primers.

### Example 5

### Second Strand Synthesis and PCR Amplification of cDNA

The cDNA was amplified by PCR. To the cDNA, Takara EX-taq buffer was added at a final concentration of 1×, then dNTPs were added (final concentration: 200 micro molar each), 5′ oligonucleotide (sequence: acc tcg agc cta ggt ccg ac) and 3′ end oligonucleotide (sequence: ca gcg tcc tca agc ggc cgc), each oligonucleotide at 400 nM concentration, MgCl2 at 2.5 mM, and KCl at a final concentration of 50 mM. The components were mixed and then after 5 minutes at 94° C., samples were incubated for 30 seconds at 94° C., 30 seconds at 58° C., and 1.5 minutes at 68° C., for 30 cycles.

This produced 5′-end cDNA that were complete and could be blunted and cloned following standard techniques into a plasmid vector (see Sambrook et al., supra, for general information about molecular cloning and sequencing).

### Example 6

### Application for RACE Experiment

The capped RNA was prepared as in Example 2, with the only difference that the RNA oligonucleotide had a different sequence as described below. By using the process of Example 2, followed by PCR, it was possible to amplify 5′-ends by RACE. The experiment was performed as follows: 500 ng of total RNA from liver was subjected to ONE-Tube oligo-capping, followed by the removal of the unreacted oligoribonucleotides, and reverse-transcription with random primers. The 5′ ends were amplified with a gene-specific primer having a sequence of:

### TTGGAGAGAGGGTTTCGACGAGTCA

and a primer complementary to the oligo-cap having a sequence of:

### CGACTGGAGCACGAGGACACTGA.

### Example 7

### Application for 454 Sequencing or Other Matrix

The cDNA was prepared as in Example 2. However, the oligonucleotides were prepared and designed in order to have the different adaptors at the 3′ and 5′-end of the RNA, respectively:

### Adaptor A:

### CCATCTCATCCCTGCGTGTCCCATCTGTTCCCTCCCTGTCTCAG;

### Adaptor B:

/5BioTEG/CCUAUCCCCUGUGUGCCUUGCCUAUCCCCUGUUGCGUGUCUCAG Adaptor B was used as an “oligo-capping” sequence, and Adaptor A was used conjugated to a oligo-random primer for the first strand synthesis. After the first strand synthesis, the material was passed through a C1-4B spin column to separate the excess of unreacted primer. Subsequently, the sample was subjected to the emulsion-PCR and then sequencing reactions as described for the 454-Life Science sequencing instrument (Margulies et al, Nature, 2005; 437(7057): 376-380, hereby incorporated herein by reference). This resulted in identifying hundreds of thousands sequences in a single run.

### Example 8

### Application for 5′-End Sequencing Tags

The cDNA was obtained as in Example 2 and in the subsequent examples, and the sample was processed until the second strand cDNA was obtained by using standard protocols known to a person skilled in the art, such as the one described in Kodzius et al., Nat. Methods. 2006 March; 3(3): 211-22, hereby incorporated herein by reference. The cDNA was then cleaved with MmeI and followed by addition of a second linker, amplification, purification and production of concatamers. Detailed protocols for such procedures are described elsewhere, such as in Kodzius et al., Nat. Methods. 2006 March; 3(3):211-22, hereby incorporated herein by reference. These sequencing tags could then be further used for sequencing and then identifying gene borders (like in Carninci et al, Science. 2005 Sep. 2; 309(5740):1559-63, hereby incorporated herein by reference) and expression profiling, or as a promoter of the genes (Harbers and Carninci, Nat. Methods, 2005 July; 2(7): 495-502, hereby incorporated herein by reference).

### Example 9

### Sequencing DNA Bound to Solid Surface

The cDNA was obtained as in Example 2 and the subsequent examples, and the sample was processed until the first strand cDNA was obtained by using standard protocols known to a person skilled in the art, such as those described in Kodzius et al., Nat. Methods. 2006 March, 3(3):211-22. Subsequently, the nucleic acids were attached to a solid-phase matrix as in the US patent application Nos. 20060012793, 20060012784, and 20060008824, and instruments based on such technology.

1. A method for preparing an RNA template for analysis, hybridization, and sequencing, comprising the steps of:
A) removing a phosphate group at the 5′-end from a first group of RNA molecules among a plurality of RNA molecules in a sample,
B) converting a CAP structure at the 5′-end of RNA into a phosphate group for a second group of RNA molecules among the plurality of RNA molecules,
C) building a functional group covalently to RNA molecules having the phosphate group at their 5′-end for the second group of RNA molecules within the plurality of RNA molecules to produce modified RNA molecules,
D) binding the modified RNA molecules in the second group of RNA molecules to a solid support using the functional group, and
E) removing non-modified RNA molecules from modified RNA molecules bound to the solid support.

2. The method of claim 1, wherein the plurality of RNA molecules are obtained from a biological material and are total RNA or mRNA.

3. The method of claim 1, wherein the RNA molecules having a Cap structure are full-length mRNA molecules.

4. The method of claim 1, wherein the 5′-end phosphate group of an RNA molecule is removed by means of an enzymatic activity of phosphatase.

5. The method of claim 4, wherein the phosphatase is one out of bacterial alkaline phosphatase, calf intestine alkaline phosphatase, shrimp alkaline phosphatase, antarctic phosphatase, or any mixture thereof.

6. The method of claim 1, wherein the CAP structure is converted into a phosphate group by means of an enzymatic activity, said a pyrophosphatase.

7. The method of claim 6, wherein the pyrophosphatase is tobacco acid pyrophosphatase.

8. The method of claim 1, wherein the functional group comprises one or more oligonucleotides or modified oligonucleotides.

9. The method of claim 7, wherein the nucleotides are ribonucleotides, desoxyribonucleotides or a mixture of ribonucleotides and desoxyribonucleotides.

10. The method of claim 8, wherein the nucleotides contained in the functional group are in part double-stranded.

11. The method of claim 1, wherein the functional group is comprised of one or more nucleotides and one or more binding molecules.

12. The method of claim 11, wherein the binding molecule is one or more reactive group, amino group, biotin, digoxigenin, or any mixture thereof.

13. The method of claim 1, wherein the functional group is covalently bound to phosphorylated RNA by means of a ligase.

14. The method of claim 13, wherein the ligase is one out of T4 RNA ligase, thermo phage single-stranded DNA ligase, T4 DNA ligase, E. coli DNA ligase, Taq DNA ligase, or any mixture thereof.

15. The method of claim 1, wherein the solid support has a planar surface or is a round shaped bead.

16. The method of claim 15, wherein the solid support is made of acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polyactic acid, polyorthoesters, functionalized silane, polypropylfumerate, collagen, glycosaminoglycans, polyamino acids, or any combination thereof.

17. The method of claim 1, wherein the modified RNA is bound to the solid support by means of hybridization of the functional group to an oligonucleotide on the solid support having complementary sequence to regions within the functional group.

18. The method of claim 1, wherein the modified RNA is bound to the solid support by means of biotin, and biotin is bound to avidin or streptavidin bound to the solid support.

19. The method of claim 1, wherein the modified RNA is bound to the solid support by means of digoxigenin, and digoxigenin is bound to a digoxigenin-binding group bound to the solid support.

20. A method for preparing an RNA template for analysis, hybridization, and sequencing, comprising the steps of:
A) covalently building a functional group at a 5′-end of RNA molecules having a phosphate group at their 5′-end to prepare a group of modified RNA molecules among a plurality of RNA molecules in a sample,
B) binding the modified RNA molecules to a solid support using the functional group, and
C) removing non-modified RNA molecules from modified RNA molecules bound to the solid support.

21. A method for preparing a DNA template for analysis, hybridization, and sequencing comprising the steps of:
A) removing a phosphate group at a 5′-end of a group of RNA molecules among a plurality of RNA molecules in a sample,
B) converting a CAP structure at the 5′-end of a second group of RNA molecules among the plurality of RNA molecules into a phosphate group,
C) covalently building a functional group at a 5′-end of RNA molecules having a phosphate group at their 5′-end, said preparation of modified RNA molecules within a plurality of RNA molecules,
D) hybridizing at least one primer to RNA molecules within the plurality of RNA molecules,
E) preparing a single-stranded DNA having in part or entirely a sequence complementary to an RNA molecule bound to a primer and contained in the plurality of RNA molecules,
F) removing RNA molecules from single-stranded DNA molecules, and
G) binding the single-stranded DNA to a solid support using the functional group or information derived therefrom.

22. The method of claim 21, wherein the single-stranded DNA is prepared from an RNA template by means of a reverse transcriptase.

23. The method of claim 22, wherein the reverse transcriptase lacks in part or entirely an RNase H activity.

24. The method of claim 22, wherein the reverse transcriptase in one out of M-MuLV Reverse Transcriptase, H Minus M-MuLV Reverse Transcriptase, Superscript II, Superscript III, AMV Reverse Transcriptase, MonsterScript, Expand Reverse Transcriptase, or any mixture thereof.

25. The method of claim 21, wherein the RNA is removed by alkali treatment or an RNase.

26. The method of claim 25, wherein the RNase is RNase H.

27. The method of claim 21, wherein the functional group is in part or entirely made from desoxyribonucleotides, wherein the functional group or parts thereof remain bound to the DNA molecule after the removal of the RNA template to form a region of double-stranded DNA within the DNA molecule, wherein the functional group contains a binding molecule, and wherein the binding molecule is used to bind DNA molecule to solid support.

28. The method for preparing a DNA template for amplification, analysis, hybridization, and sequencing comprising the steps of:
A) removing the phosphate group at the 5′-end from a group of RNA molecules contained in a plurality of RNA molecules,
B) converting a CAP structure at the 5′-end of RNA into a phosphate group for a group of RNA molecules contained in a plurality of RNA molecules,
C) covalently binding a functional group to RNA molecules having a phosphate group at their 5′-end, said preparation of modified RNA molecules within a plurality of RNA molecules,
D) hybridizing at least one primer having a functional group to RNA molecules within the plurality of RNA molecules,
E) preparing a single-stranded DNA having in part or entirely a sequence complementary to an RNA molecule bound to a primer and contained in the plurality of RNA molecules,
F) removing RNA molecules from single-stranded DNA molecules,
G) synthesizing a region of double-stranded DNA within the DNA molecule by the use of a DNA polymerase,
H) connecting the 5′-end and 3′-ends of the in part double-stranded DNA molecule by the use of a DNA ligase to form a single-stranded circular DNA molecule,
I) amplifying the single-stranded circular DNA molecule by a rolling circle method to create a linear single-stranded DNA molecule, and
J) binding the linear single-stranded DNA molecule to a solid support.

29. The method of claim 28, wherein sequence information from the sense and antisense strands from a bi-directional template is obtained in one or more sequence reaction.

30. The method of claim 28, wherein one or more sequencing reactions are performed on a linear single-stranded DNA molecule, from a bi-directional template, in a defined location on the solid support, and wherein the defined location is used to link different sequencing reads to the same liner single-stranded DNA molecule.

31. A method for preparing a DNA template for analysis, hybridization and sequencing, comprising the steps of:
A) hybridizing at least one primer having a functional group to a group of RNA molecules within a plurality of RNA molecules,
B) preparing a single-stranded DNA having in part or entirely a sequence complementary to an RNA molecule to which the primer is bound and which is among the plurality of RNA molecules, so that the primer and the functional group are incorporated into the single-stranded DNA molecule,
C) removing the RNA molecule from the DNA molecule,
D) binding the DNA molecule to a solid support using the functional group, and
E) removing DNA molecules having no functional group from DNA molecules having a functional group and bound to the solid support.

32. The method of claim 31, wherein the primer has in part a random sequence.

33. The method of claim 13, wherein the primer contains a region comprising an oligo-dT stretch.

34. The method of claim 31, wherein the primer contains a region of defined sequence complementary to a known sequence of an RNA.

35. The method of claim 31, wherein the primer contains in part a region that does not hybridize to the RNA template.

36. The method of claim 31, wherein the single-stranded DNA is prepared from an RNA template by means of a reverse transcriptase.

37. The method of obtaining one or more sequencing reads for a template on a solid support to obtain in part or the entire sequence information contained in the DNA molecule.

38. The method of claim 37, wherein the template is a modified RNA attached to a solid support.

39. The method of claim 37, wherein the template is DNA attached to a solid support.

40. The method of claim 1 to perform a diagnostic assay.

41. The method of claim 1 to prepare reagents for a kit.


SEQUENCE LISTING (7 sequences)

SEQ ID NO: 1 - RNA, 28 nt, Artificial
  primer
agagagagaccucgagccuagguccgac

SEQ ID NO: 2 - DNA, 20 bp, Artificial
  5′ oligonucleotide
acctcgagcctaggtccgac

SEQ ID NO: 3 - DNA, 20 bp, Artificial
  3′ end oligonucleotide
cagcgtcctcaagcggccgc

SEQ ID NO: 4 - DNA, 25 bp, Artificial
  a gene-specific primer
ttggagagagggtttcgacgagtca

SEQ ID NO: 5 - DNA, 23 bp, Artificial
  a primer complementary to the oligo-cap
cgactggagcacgaggacactga

SEQ ID NO: 6 - DNA, 44 bp, Artificial
  Adaptor A conjugated to a oligo-random primer for the first strand synthesis
ccatctcatccctgcgtgtcccatctgttccctccctgtctcag

SEQ ID NO: 7 - RNA, 44 nt, Artificial
  Adaptor B used as an “oligo-capping” sequence
ccuauccccugugugccuugccuauccccuguugcgugucucag